SEQUENCE LISTING (38 sequences)

SEQ ID NO: 1 - DNA, 5243 bp, Artificial Sequence
  Description of Artificial Sequence/Note = synthetic construct
gatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccgctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgaactgtacaagtccggactcagatccagaatgaatcgcacggcatacaccgtaggagctttgcttctcctcctgggaaccctactgccagcagctgaagggaaaaagaaagggtcccaaggagccatcccacctcctgacaaggctcagcacaatgactccgagcagacccagtccccaccacaacctggctccaggacccgggggcggggccaggggcggggcaccgccatgcctggagaggaggtgcttgagtccagccaagaggccctgcatgtgacagagcgcaaatacctgaagcgagattggtgcaaaactcagcccctgaagcagaccatccatgaggagggctgcaacagccgcactatcatcaatcgcttctgttacggccagtgcaactccttctacatccccaggcatatccgaaaagaggaaggctcctttcagtcttgctccttctgcaagcccaagaaattcaccaccatgatggtcacactcaactgtcctgagctacagccacccaccaagaagaaaagagtcacacgcgtgaagcagtgtcgttgcatatccatcgacttggattaag

SEQ ID NO: 2 - DNA, 3319 bp, Artificial Sequence
  Description of Artificial Sequence/Note = synthetic construct
gaaagcgcaggccccgaggacccgccgcactgacagtatgagccgcacagcctacacggtgggagccctgcttctcctcttggggaccctgctgccggctgctgaagggaaaaagaaagggtcccaaggtgccatccccccgccagacaaggcccagcacaatgactcagagcagactcagtcgccccagcagcctggctccaggaaccgggggcggggccaagggcggggcactgccatgcccggggaggaggtgctggagtccagccaagaggccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggattaagccaaatccaggtgcacccagcatgtcctaggaatgcagacccaggaagtcccagacctaaaacaaccagattcttacttggcttaaacctagaggccagaagaacccccagctgcctcctggcaggagcctgcttgtgcgtagttcgtgtgcatgagtgtggatgggtgcctgtgggtgtttttagacaccagagaaaacacagtctctgctagagagcacttcctattttgtaaacctatctgctttaatggggatgtaccagaaacccacctcaccccggctcacatctaaaggggcggggccgtggtctggttctgactttgtgtttttgtgccctcctggggaccagaatctcctttcggaatgaatgttcatggaagaggctcctctgagggcaagagacctgttttagtgctgcattcgacatggaaaagtccttttaacctgtgcttgcatcctcctttcctcctcctcctcacaatccatctcttcttaagttgacagtgactatgtcagtctaatctcttgtttgccagggttcctaaattaattcacttaaccatgatgcaaatgtttttcatttggtgaagacctccagactctgggagaggctggtgtgggcaaggacaagcaggatagtggagtgagaaagggagggtggagggtgaggccaaatcaggtccagcaaaagtcagtagggacattgcagaagcttgaaaggccaataccagaacacaggctgatgcttctgagaaagtcttttcctagtatttaacaaaacccaagtgaacagaggagaaatgagattgccagaaagtgattaactttggccgttgcaatctgctcaaacctaacaccaaactgaaaacataaatactgaccactcctatgttcggacccaagcaagttagctaaaccaaaccaactcctctgctttgtccctcaggtggaaaagagaggtagtttagaactctctgcataggggtgggaattaatcaaaaacctcagaggctgaaattcctaatacctttcctttatcgtggttatagtcagctcatttccattccactatttcccataatgcttctgagagccactaacttgattgataaagatcctgcctctgctgagtgtacctgacagtagtctaagatgagagagtttagggactactctgttttaacaagaaatattttgggggtctttttgttttaactattgtcaggagattgggctaaagagaagacgacgagagtaaggaaataaagggaattgcctctggctagagagtagttaggtgttaatacctggtagagatgtaagggatatgacctccctttctttatgtgctcacttgaggatctgaggggaccctgttaggagagcatagcatcatgatgtattagctgttcatctgctactggttggatggacataactattgtaactattcagtatttactggtaggcactgtcctctgattaaacttggcctactggcaatggctacttaggattgatctaagggccaaagtgcagggtgggtgaactttattgtactttggatttggttaacctgttttcctcaagcctgaggttttatatacaaactccctgaatactctttttgccttgttacttctcagcctcctagccaagtcctatgtaatatggaaaacaaacactgcagacttgagattcagttgccgatcaaggctctggcattcagagaacccttgcaactcgagaagctgtttttgatttcgtttttgttttgaaccggtgctctcccatctaacaactaacsaggaccatttccaggcgggagatattttaaacacccaaaatgttgggtctgatttccaaacttttaaactcactactgatgattctcacgctaggcgaatttgtccaaacacatagtgtgtgtgttttgtatacactgtatgaccccaccccaaatctttgtattgtccacattctccaacaataaagcacagagtggatttaattaagcacacaaatgctaaggcagaattttgagggtgggagagaagaaaagggaaagaagctgaaaatgtaaaaccacaccagggaggaaaaatgacattcagaaccaccaaacactgaatttctcttgttgttttaactctsccacaagaatgcawtttcgttaatggagatgacttaagttggcagcagaaatcttcttttaggagcttgtcccccaktyttgcacataagtgcagatttgccccaagtaaagagaatttcctcaacactaacttcacggggataatcaccacctaamcrcccttaaagcawatcactagccaaagaggggaatatctgttcttcttactgtgcctatattaagactagtacaaatgtggtgtgtcttccaactttcaktgaaaatgccatatctataccatattttattcgagtcactgatgatgtaatgatatattttttcattattatagtagaatatttttatggcaagawatttgtggtcttgatcatacctattaaaataatgccaaacaccaaatatgaattttatgatgtacactttgtgcttggcattaaaagaraaaaacacacaccggaattccagctgagcgccggtcgctaccattaccagttggtctggtgtcaaaagccgaattctgcagatatccatcacactggcggccgctcgagcatgcatctagagggccc

SEQ ID NO: 3 - DNA, 3795 bp, Artificial Sequence
  Description of Artificial Sequence/Note = synthetic construct
gcggccgcgagctctaatacgactcactatagggcgtcgactcgatcagatacatagtaacccaagctgacacaagcttagaacctacagtcggagcaggagttgaatgtcacattatcagctccaaacttgaacctgctccaaagtattaagttaatgtcagaaaaacaatgacatttaagaatatttttaatgaaacattcaattatcttggttcgatgctagccttagggttggatggccctcacttgccagaagttgtcctttaaaggagatccatcttaggctgctttttgtctcttagagataattggtctagataatgataccaacttgtctggttccttggagatgaaggttatattaaaaaggttatgtcaatatgcacttagtggttgccacatgcaatactggtattcagcggacagaaaatggatgcttccttgctgttcttgtgcagcaaaccttaaccatggggcagaggaaaccccagggtagctgccatgcctggaagagacattatgtatttgaaactgttctcatttgaaaagaaagccttcaatgctttaataactcttggtgtgccccaggccagcaagtgttccaggcttttagctgggtgggaaggctggctgactgagttaggatcttcatattaatgctttcccagaggactgtgtccagggatactgccccaggagaatcctgacagcctgctgcctctctttcccttttccgcctgtctgccctgtcttttctgaacaacaccgcctctgaaaagtctcctcttctcttatttgctttgtttacctcatgttcctgtctctgtatgtttcttctcccaccaggtgggagatcatgcttagacttattgctttatttatttataatgtatttatttataatttatttatttattaaatgttatatgcccttgccatatacgagtcatatcaaggtccacatttgctcacagttcattggcatcaattctattcttatgaattgaaatattcccgtacttactctctattgtgcccatttttctaccttacacacactctctcttcttcttctttcttcttcttcttcttcttcttcttcttcttcttcttcttcttcttcttcttcttcttctttttctttctttctttcttcttcttcttcttcttcttcttcttcttcttcttcttcttcttcttcttcttttctctctctctctctctctctctctctctctctctctctctctctctctccacatgtggcttgaaagcagaaggactgtttggggaaatgacacagtaaagcagcagggggaggcaaatgtgaacaaggtgaggtgacagatatgcatgaaaatccacaatgaaactccgtcttgtacaccaacttaaaaattaaagccagagaaattaaagacctacctggtcaattaatcagacaaaaaaaaattctattcatacatacagtcacatagatgggtaatgtattttaccacttagaaaggttgaaaagtggggtctggagaaatggctcatcagctaagaacactttctgttcttccaagcgttctgagttcagttgccagcactcacattgggggctcacaactgcctataattccagctttaggagttctgggtgttttattgccctccctaggcacacacacggattacacagacacacacacacacacacacacacacacacacacacacacaagttgttatatcatggcagaaagaatgataccagccatctttatcctcttggccttccgtacatccctctttttaggttctttttttttttgacaggtttcctgggctttttccaatactggaacagtgaaaagtctcatgtcaaattcaaggataaatacagttaagtgagcattaaaaaaagtcacatgcaattgtgtcaggagccagtaaggaattctaataggagctggttcaaaagagagacgggtcctgactgagtttaaagcttggcaaattcactgtgtgacctgtgtcgaattactcagtttgatggctgagagaataatggaaataatagtatctaatggctggtgatactgttagaagtcagtgcaactgaagtgtgtgttgagtacagtgtgttaagtgtaattattgatttttactaaataactttcttattgtctgtgtccccctctctttgtcctttgtctagaatgaatcgcaccgcatacactgtgggagcgttgcttctcctcctggggaccctactgccaacagctgaggggaaaaagaaaggttcccaaggagccattccgcctcctgacaaggctcagcacaatgactctgagcagacccagtccccaccacaacctggctccaggacccgggggcggggccaggggcggggcaccgccatgcctggagaggaggtgcttgagtccagccaagaggccctgcacgtgacagagcgcaagtatctgaagcgagattggtgcaaaactcagcccctgaagcagaccatccacgaggagggctgcaacagccgcactatcatcaaccgcttctgttatggccagtgcaactccttctacatccccaggcacatccgaaaggaggaagggtcctttcagtcttgctccttctgcaagcccaagaagttcaccaccatgatggtcacactcaactgtcctgagctacagccacccaccaagaagaaaagggtcacacgcgtgaagcagtgccgttgcatatccatcgacttggattaagtcaaagcgggcacattcagcctgtcatagccatgctgagagagccacacccaaaccacccgattcctacttggcttaaacctagaggccagaagaaccagcagttgcttcctggctggaggctgcttatgcatagtgtatgcgcatgagtgtgcatgggtgcctgtgggtgtttccaaacaccagccggaaacagcctttgctagaaggcacttcctgttactctgcttcagatggtcggaaatgcccacaccactggacccaaacatccacaggggcagggctgtagttggctttgtcattgtgttccatgtgcctcctgggcaccaggatttcacttgagaatgaatactaatgggggaggtaactctgagggctgcattagactcggaactgttcagtgctcgccctatgctcccatagcccatccctttctttgctctccctgacatctcagtcgtagcccatgttcctaaattaattcacttgaccgcgggtgtaagtcttttgtcttgtgaagaaccttcagaatgtggggagacacgtggtgatggcaaacgggacagaggactgacgcaggaacggtcaggctgaggaccagtctgggccagtgacattcagtagtgagatgtctagagtttaaaagttgtttcccaaaacaatattagtcttgtttttagcaaaagggttttcctgatatttaaaagaacccagacacacagaggaaaaatataatcagcaaaaaaacaaaacaaaacaaaataacacaaacaataacaacaacaacaaacaaaaacccaattctctgtgccagcttctgtgacctactgatactagctgtaactgatactagctgttaagggtgaaatgctgaccactcctgttttaagaaccaagtgaaattaaaaaagaaaatgtggcctcctactttactttgcctctctgaagtacaactgagagccttgttcactggggtaagagaaggcaaatcctcctaagcttagtttcgctggattaacattgcttgtccgccg

SEQ ID NO: 4 - DNA, 3820 bp, Artificial Sequence
  Description of Artificial Sequence/Note = synthetic construct
acctggggagccagagcaccgcagtagcgcactttccttcgtgttcttcccgcgtcgagcccgagtggctccggccgcggtcgcacgcaacgccacgcgtccacagcgaaggacttgaggatccactgaggtgacagaatgaatcgcacggcatacaccgtaggagctttgcttctcctcctgggaaccctactgccagcagctgaagggaaaaagaaagggtcccaaggagccatcccacctcctgacaaggctcagcacaatgactccgagcagacccagtccccaccacaacctggctccaggacccgggggcggggccaggggcggggcaccgccatgcctggagaggaggtgcttgagtccagccaagaggccctgcatgtgacagagcgcaaatacctgaagcgagattggtgcaaaactcagcccctgaagcagaccatccatgaggagggctgcaacagccgcactatcatcaatcgcttctgttacggccagtgcaactccttctacatccccaggcatatccgaaaagaggaaggctcctttcagtcttgctccttctgcaagcccaagaaattcaccaccatgatggtcacactcaactgtcctgagctacagccacccaccaagaagaaaagagtcacacgcgtgaagcagtgtcgttgcatatccatcgacttggattaagtcaaagggggcacattcagcctgtcatagccatgccgagagccacacccaaaccacccgattcctacttggcttaaacctagaggccagaagaaccagcagttgcttcctggctggaggctgcttatgcatagagtatgcgcatgagtgtgcatgggtacctgtgggtgtttccaaacaccagcggaaacagcctctgcaggaaggcacttcctgttactgtgcttcagatggtcggaaatgctcacaccactggacccaacaccacaggggcagggctgtagatgactttgaccttgtgttccattggcctcctgggcaccaggatttcatttgagaatgaatactaacggaggaggtaactctgagggccgcattagactcggaacagtttgttcgtgctctcccacaacccattcctttctttgctctccctgaccttagtccatgttcttaaattaattcacttgatgtgagtgtaaatttctttcgtcttgtgaagaaccttcagagtgtggggagacaagtgataaaggcaaacagaacaggggattgacacaggagcattgagactgaggaccagtctggccagtgaaattcagtagcaagatgttcagagtttaaagattgttcccccccaaacaatatgagtcttgttttagcaaaggggctttactgatatttaaaagaacccagacagacagaggagaaatataatcagcaaaaaaaccaattctctgtgccggtatctgtgacctactgacaatatctgtaatccaatgttaagggtgaaatattgaccacttctgttttaagaaccaagtgaaaggaaaaaaaaaatatggccttctacttactttgcctctcaggaggatgactgagagcgttgttcgctagggtaagaaagacaaaacctcctaggcttagttttgctggattatcattgctttcccatcattcctgaaaaaatgcttcagagatgcagaaccttccaataaaatcgtgcttttcttgagaccatttgccagtaagggtcagtgttagacgagagagctgtctgctgcatgtgagttagacatgtctggggcttcttctgtttggcttttgttataggagagaaccagagatgagagagctgatgagagaacagagacagagagagagagggccaatcccttagggaagcactagggtatattaacaggccacctacacccaatggatctatgtgacattgtaatcattatgcctactatggatgctgtcctctgaatacacatggctgcccaatgtctacttagcatctatgtaagggcccagagaaaggtgactgggtcttggtacattttggtttggctaagcaatactcttttaagactgacattctagctataaatgccccagatactttttttgccttttcctctcagagcgactagtcaagtgatatgtcatttggaaggcagacattcactgcccatcaaagataccacagtcaaagaaccattgggagtaaagaaactttttgttttggtctagcccacccgcccatgtaacatcgaaacaggaaccatattacaaggcaaaagctatcttgaattcccaaaacactgggtctaattttgaaagtttaaaagtcactggtgatgactccacagtaagtgaacttgtgcgagcatagccgtgagtttcatttgtactgcgtgctccttcactgaatctttgaggcttccatatccatagccacatagtcacagggtggatttgattaggcccacacatacaaaggtgggtttggagggtggtgaagagggaaaaataagagaggatgaagatgaaaatatagacccacaccagagaggaaaaatgaccctcggtgctgaaaaacactgtgtcccatcttaattctgccacaaacatgcagtcttgctaaaaatcaacaacaacaataataaaaatgtttggcagccacagttacctttaggagcttgtaccacagtctctcttgtaagctggatttagatttggttcttgacgattgcctcaaaattaacttctttgaaacgatcagcagcataagtgccctaaaagcacatcactggccaacggctgggacgtctgccttccttgccgtgcctagatcaagaccatcagaaaatgtgtccgctgccgtttattggagatgccccgtctgtcgctgattctggacgcaccagcgatgcaaggatggacactttctccaacattgtagtagaaccaattttttttggcaagctttgttgcagtctccaccttacctgttaaataatgccagaaaccaaatatgaatcttacggcattcaattgtgcttggcactgaaagaggaaagccacacaccagataagtctgagtgcccctttgccattgtactcttcaaagtgagaaacctggaggaaggatagtctccatgtggaatgtgaataagcaaaagagttatggttatttaatgtaattaggaattctaggtccttcggttactgtgatttcgaatgttttctttctctgttttatacgacagcctctgagttggggcaaagaagaaacaggccgttgtatgttgctagagactttcgtcaggtcagggggacacacagtcttgtcacatatgaagagatgttaccaagtcaacgacaagccttattttttaacgttgaatgttccttaaaggctgacacttctgaagcaatgttaggaaagactttaaatgttattttgagagacttctgtgcgtatacaagcagataatgacggcatgttcagacaagcagaacatttctaaacgagaagtccgagctgaacgactgaaaagagattcctcgccatattgaatatcatctacattgtgtatttaatatactttaatcattttgaaacaacgaaggattatgcaggctatgacggaactactaccttgctatggatgagggttgggcaggatttaatggtctcatagaagctaatttggcttaaagttttatgaatctgtaactagaattttattttcaccctaataacattctatataacctttgccaaaaaagcaatcaataaattaacctcttctttctgtggcaaaaaaaaaaaaaa

SEQ ID NO: 5 - DNA, 5168 bp, Artificial Sequence
  Description of Artificial Sequence/Note = synthetic construct
gatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgaactgtacaagtccggactcagatccagaatgaatcgcacggcatacaccgtaggagctttgcttctcctcctgggaaccctactgccagcagctgaagggaaaaagaaagggtcccaaggagccatcccacctcctgacaaggctcagcacaatgactccgagcagacccagtccccaccacaacctggctccaggacccgggggcggggccaggggcggggcaccgccatgcctggagaggaggtgcttgagtccagccaagaggccctgcatgtgacagagcgcaaatacctgaagcgagattggtgcaaaactcagcccctgaagcagaccatccatgaggagggctgcaacagccgcactatcatcaatcgcttctgttacggccagtgcaactccttctacatccccaggcatatccgaaaagaggaaggctcctttcagtcttgctccttctgcaagcccaagaaattcaccaccatgtaagtcgcttcgacttggattaag

SEQ ID NO: 6 - DNA, 5166 bp, Artificial Sequence
  Description of Artificial Sequence/Note = synthetic construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattcaatgaatcgcacggcatacaccgtaggagctttgcttctcctcctgggaaccctactgccagcagctgaagggaaaaagaaagggtcccaaggagccatcccacctcctgacaaggctcagcacaatgactccgagcagacccagtccccaccacaacctggctccaggacccgggggcggggccaggggcggggcaccgccatgcctggagaggaggtgcttgagtccagccaagaggccctgcatgtgacagagcgcaaatacctgaagcgagattggtgcaaaactcagcccctgaagcagaccatccatgaggagggctgcaacagccgcactatcatcaatcgcttctgttacggccagtgcaactccttctacatccccaggcatatccgaaaagaggaaggctcctttcagtcttgctccttctgcaagcccaagatattcaccaccatgtaaggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 7 - DNA, 5130 bp, Artificial Sequence
  Description of Artificial Sequence/Note = synthetic construct
gatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgaactgtacaagtccggactcagaatgagggctcagcacaatgactccgagcagacccagtccccaccacaacctggctccaggacccgggggcggggccaggggcggggcaccgccatgcctggagaggaggtgcttgagtccagccaagaggccctgcatgtgacagagcgcaaatacctgaagcgagattggtgcaaaactcagcccctgaagcagaccatccatgaggagggctgcaacagccgcactatcatcaatcgcttctgttacggccagtgcaactccttctacatccccaggcatatccgaaaagaggaaggctcctttcagtcttgctccttctgcaagcccaagaaattcaccaccatgatggtcacactcaactgtcctgagctacagccacccaccaagaagaaaagagtcacacgcgtgaagcagtgtcgttgcatatccatcgacttggattaag

SEQ ID NO: 8 - DNA, 5054 bp, Artificial Sequence
  Description of Artificial Sequence/Note = synthetic construct
gatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgaactgtacaagtccggactcagaatgagggctcagcacaatgactccgagcagacccagtccccaccacaacctggctccaggacccgggggcggggccaggggcggggcaccgccatgcctggagaggaggtgcttgagtccagccaagaggccctgcatgtgacagagcgcaaatacctgaagcgagattggtgcaaaactcagcccctgaagcagaccatccatgaggagggctgcaacagccgcactatcatcaatcgcttctgttacggccagtgcaactccttctacatccccaggcatatccgaaaagaggaaggctcctttcagtcttgctccttctgcaagcccaagaaattcaccaccatgtaagtcgcttcgacttggattaag

SEQ ID NO: 9 - DNA, 5031 bp, Artificial Sequence
  Description of Artificial Sequence/Note = synthetic construct
gatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgaactgtacaagtccggactcagaatgagggctcagcacaatgactccgagcagacccagtccccaccacaacctggctccaggacccgggggcggggccaggggcggggcaccgccatgcctggagaggaggtgcttgagtccagccaagaggccctgcatgtgacagagcgcaaatacctgaagcgagattggtgcaaaactcagcccctgaagcagaccatccatgaggagggctgcaacagccgcactatcatcaatcgcttctgttacggccagtgcaactccttctacatccccaggcatatccgaaaagaggaaggctcctttcagtcttgctccttctgcaagcccaagatattcaccaccatgt

SEQ ID NO: 10 - DNA, 50 bp, Artificial Sequence
  Description of Artificial Sequence/Note = synthetic construct
ccggggacgaggacagctgtaattacctgctcctgtcgacattaatggcc

SEQ ID NO: 11 - DNA, 29 bp, Artificial Sequence
  Description of Artificial Sequence/Note = synthetic construct
cgggatccagaatgaatcgcacggcatac

SEQ ID NO: 12 - DNA, 31 bp, Artificial Sequence
  Description of Artificial Sequence/Note = synthetic construct
gcggatccttaatccaagtcgatggatatgc

SEQ ID NO: 13 - DNA, 27 bp, Artificial Sequence
  Description of Artificial Sequence/Note = synthetic construct
taagtcgcttcgacgtacattcagcga

SEQ ID NO: 14 - DNA, 27 bp, Artificial Sequence
  Description of Artificial Sequence/Note = synthetic construct
aggaattcaatgaatcgcacggcatac

SEQ ID NO: 15 - DNA, 32 bp, Artificial Sequence
  Description of Artificial Sequence/Note = synthetic construct
acgggatccttacatggtggtgaatacttggg

SEQ ID NO: 16 - DNA, 53 bp, Artificial Sequence
  Description of Artificial Sequence/Note = synthetic construct
gtacaagtccggactcagaatgagggcttcaggcctgagtcttactcccgagt

SEQ ID NO: 17 - DNA, 53 bp, Artificial Sequence; identical to SEQ ID NO: 16
  Description of Artificial Sequence/Note = synthetic construct

SEQ ID NO: 18 - DNA, 53 bp, Artificial Sequence; identical to SEQ ID NO: 16
  Description of Artificial Sequence/Note = synthetic construct

SEQ ID NO: 19 - DNA, 5268 bp, Artificial Sequence
  Description of Artificial Sequence/Note = synthetic construct
gatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccgctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgaactgtacaagtccggactcagatccagaatgaatcgcacggcatacaccgtaggagctttgcttctcctcctgggaaccctactgccagcagctgaagggaaaaagaaagggtcccaaggagccatcccacctcctgacaaggctcagcacaatgactccgagcagacccagtccccaccacaacctggctccaggacccggggacgaggacagctgtaattaccgggggcggggccaggggcggggcaccgccatgcctggagaggaggtgcttgagtccagccaagaggccctgcatgtgacagagcgcaaatacctgaagcgagattggtgcaaaactcagcccctgaagcagaccatccatgaggagggctgcaacagccgcactatcatcaatcgcttctgttacggccagtgcaactccttctacatccccaggcatatccgaaaagaggaaggctcctttcagtcttgctccttctgcaagcccaagaaattcaccaccatgatggtcacactcaactgtcctgagctacagccacccaccaagaagaaaagagtcacacgcgtgaagcagtgtcgttgcatatccatcgacttggattaag

SEQ ID NO: 20 - DNA, 22 bp, Artificial Sequence
  Description of Artificial Sequence/Note = synthetic construct
tcattacatcatcagtgactcg

SEQ ID NO: 21 - DNA, 22 bp, Artificial Sequence
  Description of Artificial Sequence/Note = synthetic construct
cagatttggctcaagtaaagag

SEQ ID NO: 22 - DNA, 10 bp, Artificial Sequence
  Description of Artificial Sequence/Note = synthetic construct
agccagcgaa

SEQ ID NO: 23 - DNA, 10 bp, Artificial Sequence
  Description of Artificial Sequence/Note = synthetic construct
gaccgcttgt

SEQ ID NO: 24 - DNA, 10 bp, Artificial Sequence
  Description of Artificial Sequence/Note = synthetic construct
aggtgaccgt

SEQ ID NO: 25 - DNA, 10 bp, Artificial Sequence
  Description of Artificial Sequence/Note = synthetic construct
ggtactccac

SEQ ID NO: 26 - DNA, 10 bp, Artificial Sequence
  Description of Artificial Sequence/Note = synthetic construct
gttgcgatcc

SEQ ID NO: 27 - DNA, 26 bp, Artificial Sequence
  Description of Artificial Sequence/Note = synthetic construct
ccgctcgaggtgacagaatgaatcgc

SEQ ID NO: 28 - DNA, 51 bp, Artificial Sequence
  Description of Artificial Sequence/Note = synthetic construct
cccgttaacttaggcgtagtcgggcacgtcgtaggggtaatccaagtcgat

SEQ ID NO: 29 - protein, 429 aa, Artificial Sequence
  Description of Artificial Sequence/Note = synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRMNRTAYTVGALLLLLGTLLPAAEGKKKGSQGAIPPPDKAQHNDSEQTQSPPQPGSRTRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 30 - protein, 397 aa, Artificial Sequence
  Description of Artificial Sequence/Note = synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRMNRTAYTVGALLLLLGTLLPAAEGKKKGSQGAIPPPDKAQHNDSEQTQSPPQPGSRTRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTM

SEQ ID NO: 31 - protein, 403 aa, Artificial Sequence
  Description of Artificial Sequence/Note = synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRAQASNSMNRTAYTVGALLLLLGTLLPAAEGKKKGSQGAIPPPDKAQHNDSEQTQSPPQPGSRTRGRGQGRGTAMPGEEVLESSQEALHVTERKYLKRDWCKTQPLKQTIHEEGCNSRTIINRFCYGQCNSFYIPRHIRKEEGSFQSCSFCKPKIFTTM

SEQ ID NO: 32 - protein, 391 aa, Artificial Sequence
  Description of Artificial Sequence/Note = synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRMRAQHNDSEQTQSPPQPGSRTRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 33 - protein, 359 aa, Artificial Sequence
  Description of Artificial Sequence/Note = synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRMRAQHNDSEQTQSPPQPGSRTRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTM

SEQ ID NO: 34 - protein, 359 aa, Artificial Sequence
  Description of Artificial Sequence/Note = synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRMRAQHNDSEQTQSPPQPGSRTRGRGQGRGTAMPGEEVLESSQEALHVTERKYLKRDWCKTQPLKQTIHEEGCNSRTIINRFCYGQCNSFYIPRHIRKEEGSFQSCSFCKPKIFTTM

SEQ ID NO: 35 - protein, 308 aa, Artificial Sequence
  Description of Artificial Sequence/Note = synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRMNRTAYTVGALLLLLGTLLPAAEGKKKGSQGAIPPPDKAQHNDSEQTQSPPQPGSRTRGRGQL

SEQ ID NO: 36 - protein, 184 aa, Artificial Sequence
  Description of Artificial Sequence/Note = synthetic construct
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 37 - protein, 184 aa, Artificial Sequence
  Description of Artificial Sequence/Note = synthetic construct
MNRTAYTVGALLLLLGTLLPTAEGKKKGSQGAIPPPDKAQHNDSEQTQSPPQPGSRTRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 38 - protein, 184 aa, Artificial Sequence
  Description of Artificial Sequence/Note = synthetic construct
MNRTAYTVGALLLLLGTLLPAAEGKKKGSQGAIPPPDKAQHNDSEQTQSPPQPGSRTRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

(19) United States
(12) Patent Application Publication
(10) Pub. No.: US 2003/0078406 A1
(43) Pub. Date: Apr. 24, 2003

(54) Methods and compositions for DRM, a secreted protein with cell growth inhibiting activity

(75) Inventors: Donald G. Blair, Frederick, MD (US); Peter A. Clausen, Frederick, MD (US); Lilia Z. Topol, Frederick, MD (US); Maria Marx, L'Hay Les Roses (FR); Georges Calothy, Orsay (FR)

Correspondence Address: Mary L. Miller, Ph.D., Esq., NEEDLE & ROSENBERG, P.C., The Candler Building, Suite 1200, 127 Peachtree Street, N. E., Atlanta, GA, 30303-1811, US

(73) Assignee: The Government of the U.S.A., as represented by the Secretary, Dept. of Health & Human Services

(21) Appl. No.: 10/033,717

(22) Filed: Dec. 27, 2001

Related U.S. Application Data
(63) Continuation of application No. 09/444,066, filed on Nov. 19, 1999, now abandoned.
Application No. 09/444,066 is a continuation of application No. 09/277,407, filed on Mar. 26, 1999, now abandoned.
(60) Provisional application No. 60/079,440, filed on Mar. 26, 1998.

(51) Int. Cl. C07K 14/705; C07H 21/04; C12P 21/02; C12N 5/06

(52) U.S. Cl. 536/23.5; 530/350; 435/69.1; 435/325; 435/320.1

(57) ABSTRACT

The present invention provides an isolated nucleic acid encoding DRM protein, an isolated DRM polypeptide, and a fusion polypeptide comprising a DRM protein and a green fluorescent protein. The present invention also provides a method of arresting the growth of a cell, comprising administering to the cell an effective amount of DRM protein or an active fragment thereof; a method of inhibiting tumor cell growth, comprising administering to a tumor cell an effective amount of DRM protein or an active fragment thereof; and a method of treating a hyperproliferative cell disorder in a subject diagnosed with a hyperproliferative cell disorder, comprising administering to the subject an effective amount of DRM protein or an active fragment thereof, in a pharmaceutically acceptable carrier. In addition, the present invention provides a method of arresting growth of a cell, comprising administering to the cell an effective amount of a nucleic acid encoding a DRM protein or an active fragment thereof, a method of inhibiting tumor cell growth, comprising administering to a tumor cell an effective amount of a nucleic acid encoding a DRM protein or an active fragment thereof; and a method of treating a hyperproliferative cell disorder in a subject diagnosed with a hyperproliferative cell disorder, comprising administering to a cell of the subject, in a pharmaceutically acceptable carrier, an effective amount of a nucleic acid encoding a DRM protein or an active fragment thereof, under conditions whereby the nucleic acid is expressed in the subject's cell.

This application claims priority to U.S. patent application Ser. No. 09/277,407, filed on Mar. 26, 1999, now abandoned, which claims priority to provisional application Serial No. 60/079,440 filed on Mar. 26, 1998, both of which are hereby incorporated herein by reference in their entirety.

### BACKGROUND OF THE INVENTION

1. Field of the Invention

The present invention relates to a secreted protein with cell growth inhibiting activity. In particular, the present invention relates to the DRM protein, which is downregulated in transformed cells and which, when overexpressed, can arrest cell growth. The present invention further relates to an enhanced green fluorescent protein (EGFP)IDRM fusion, which imparts stability to the EGFP, thereby enhancing the versatility of EGFP as a research tool.

2. Background Art

Cell proliferation is determined by a complex and dynamic equilibrium between positive and negative elements signaling the cell to stay in or out of the cycle. The negative elements could be required for an efficient growth shutdown that could end with a reversible (G₀) or irreversible out-of-cycle condition (terminal differentiation, apoptosis, and senescence) (66,67). The exit from the proliferative cell cycle into a reversible quiescence (G₀) is an active process that is not yet well understood at the molecular level. Investigation of G₀-specific gene expression is an important step in studying the mechanism regulating the entrance to quiescence. The nonproliferative state (G₀) in normal cells is characterized by increased expression of a set of genes called gas (growth arrest specific) (68). These genes were originally isolated as genes whose expression was increased upon serum starvation or density inhibition (69,70). It has been shown that Gas1, when ectopically expressed, blocks the G₀-to-S phase transition of quiescent fibroblasts (69). The control of cell proliferation occurs mainly in the G1 phase.

Malignant transformation is characterized by alterations in the normal properties of cell growth, adhesion, motility and shape. The multistep nature of this process is now well defined in a number of systems, as well as the fact that genetic changes in specific genes are responsible for both positive and negative contributions to that process. Analysis of the genes involved has identified those which act positively to induce aspects of the transformed state (oncogenes) and more recently, has led to the identification of those which act to block or suppress the malignant phenotype, the so-called tumor-suppressor genes (24). The importance of these genes in maintaining the normal phenotype was first inferred by the fact that in many human tumors their functions have been lost as a consequence of deletion, rearrangement or mutations of both alleles, and indeed the most well-characterized members of this group, represented by Rb, p53, WTI and DCC, were first identified and isolated following pedigree and genetic analyses (34). The frequent physical or functional loss of these tumor-suppressor genes in specific human malignancies was strong evidence that these changes contribute to the development of the neoplastic phenotype.

Loss of function of a particular gene may occur by a variety of mechanisms, including the repression of its expression at the RNA level, and a large number of genes whose expression is repressed either in tumors or in cells transformed by positively acting oncogenes, such as v-ras, v-src or SV40 T antigen, have been identified. This group includes the retinoic acid receptor (20), α-actinin (13), maspin (44), interferon regulatory factor I (19), tropomyosin (31), as well as the DAN, 322, and rrg genes (8,26,28). Several of these were identified by subtractive hybridization or differential display techniques, which allowed the identification of RNA species whose expression was reduced in transformed cells. In gene transfer experiments, these genes exhibited tumor-suppressive and cell-growth-arrest activities, leading to the hypothesis that the reduced expression or function of certain genes was required for the expression of the transformed phenotype.

The present invention provides a nucleic acid encoding a secreted protein and a secreted protein, designated DRM, with cell growth inhibiting activity and methods for administering the nucleic acid and protein of this invention to arrest cell growth and treat hyperproliferative cell disorders. The present invention further provides an enhanced green fluorescent protein (EGFP)/DRM fusion which imparts stability to the fluorescence activity of EGFP, thus providing a much more versatile research tool than conventional EGFP.

### SUMMARY OF THE INVENTION

The present invention provides an isolated nucleic acid having the nucleotide sequence of SEQ ID NO:2 (human cDNA encoding DRM). The invention also provides an isolated nucleic acid having the nucleotide sequence of SEQ ID NO: 4 (rat cDNA sequence for DRM)

Further provided is an isolated polypeptide having the amino acid sequence of SEQ ID NO:36 (mouse DRM), an isolated nucleic acid encoding the polypeptide and an isolated nucleic acid having the nucleotide sequence of SEQ ID NO:3 (mouse cDNA encoding DRM).

In addition, the present invention provides a method of arresting the growth of a cell, comprising administering to the cell an effective amount of DRM protein or an active fragment thereof; a method of inhibiting tumor cell growth, comprising administering to a tumor cell an effective amount of DRM protein or an active fragment thereof; and a method of treating a hyperproliferative cell disorder in a subject diagnosed with a hyperproliferative cell disorder, comprising administering to the subject an effective amount of DRM protein or an active fragment thereof, in a pharmaceutically acceptable carrier.

In addition, the present invention provides a method of arresting growth of a cell, comprising administering to the cell an effective amount of a nucleic acid encoding a DRM protein or an active fragment thereof; a method of inhibiting tumor cell growth, comprising administering to a tumor cell an effective amount of a nucleic acid encoding a DRM protein or an active fragment thereof; and a method of treating a hyperproliferative cell disorder in a subject diagnosed with a hyperproliferative cell disorder, comprising administering to a cell of the subject, in a pharmaceutically acceptable carrier, an effective amount of a nucleic acid encoding a DRM protein or an active fragment thereof, under conditions whereby the nucleic acid is expressed in the subject's cell.

Further provided is a method of identifying a subject at risk of developing a hyperproliferative cell disorder, comprising measuring the amount of DRM protein or the amount of nucleic acid encoding DRM in a cell of the subject, whereby an amount of DRM protein or nucleic acid encoding DRM in a cell less than the amount of DRM protein or nucleic acid encoding DRM in a cell of a normal subject identifies a subject at risk of developing a hyperproliferative cell disorder.

The present invention additionally provides a fusion polypeptide comprising a DRM protein and a green fluorescent protein. Also provided is a green fluorescent protein having increased stability, comprising a fusion protein comprising a DRM protein amino acid sequence linked to a green fluorescent protein amino acid sequence.

An isolated nucleic acid having the nucleotide sequence of SEQ ID NO: 1 (EGFP/DRM nucleic acid) and a polypeptide having the amino acid of SEQ ID NO:29 (EGFP/DRM amino acid) is also provided.

Further provided is a method of producing a green fluorescent protein having increased stability, comprising the steps of producing a nucleic acid construct whereby a nucleic acid sequence encoding EGFP is positioned upstream and in frame with a nucleic acid encoding DRM or an active fragment thereof; placing the nucleic acid construct into an expression vector; and placing the expression vector into a cell under conditions whereby the nucleic acid of the construct will be expressed, thereby producing a green fluorescent protein having increased stability.

Various other objectives and advantages of the present invention will become apparent from the following detailed description.

### DESCRIPTION OF THE PREFERRED EMBODIMENTS

As used herein, “a” or “an” can mean multiples. For example, “a cell” can mean at least one cell.

The present invention is based on the surprising discovery of the secreted protein, DRM, which has been identified to be capable of blocking cell proliferation. The DRM protein, as well as the nucleic acid encoding the DRM protein, can be used in therapeutic applications, to treat hyperproliferative cell disorders, such as cancer. It is further contemplated that the DRM protein and its nucleic acid can be used to identify a subject at risk of developing a hyperproliferative cell disorder, such as cancer.

Thus, the present invention provides an isolated nucleic acid having the nucleotide sequence of SEQ ID NO:2, which encodes the human homologue of the DRM protein having the amino acid sequence of SEQ ID NO:37.

The present invention further provides an isolated polypeptide having the amino acid sequence of SEQ ID NO:36, which is the amino acid sequence of the mouse homologue of DRM. Also provided is an isolated nucleic acid encoding the mouse homologue of DRM and an isolated nucleic acid having the nucleotide sequence of SEQ ID NO:3, which comprises the 5′ genomic sequence and the coding sequence of the mouse homologue of DRM. The coding sequence of SEQ ID NO:3 is nucleotides 2201 through 2757. Also provided is a nucleic acid having the nucleotide sequence of SEQ ID NO:4, which encodes the rat homologue of DRM, having the amino acid sequence of SEQ ID NO:38.

“Nucleic acid” as used herein refers to single- or double-stranded molecules which may be DNA, comprised of the nucleotide bases A, T, C and G, or RNA, comprised of the bases A, U (substitutes for T), C, and G. The nucleic acid may represent a coding strand or its complement. Nucleic acids may be identical in sequence to the sequence which is naturally occurring or may include alternative codons which encode the same amino acid as that which is found in the naturally occurring sequence (61). Furthermore, nucleic acids may include codons which represent conservative substitutions of amino acids as are well known in the art.

As used herein, the term “isolated” means a nucleic acid separated or substantially free from at least some of the other components of the naturally occurring organism, for example, the cell structural components commonly found associated with nucleic acids in a cellular environment and/or other nucleic acids. The isolation of nucleic acids can therefore be accomplished by techniques such as cell lysis followed by phenol plus chloroform extraction, followed by ethanol precipitation of the nucleic acids (58). The nucleic acids of this invention can be isolated from cells according to methods well known in the art for isolating nucleic acids. Alternatively, the nucleic acids of the present invention can be synthesized according to standard protocols well described in the literature for synthesizing nucleic acids.

The nucleic acid or fragment thereof of this invention can be used as a probe or primer to identify the presence of a nucleic acid encoding the DRM polypeptide in a sample. Thus, the present invention also provides a nucleic acid, which can be the entire complementary sequence to the nucleic acid coding sequence of the DRM protein or a fragment thereof comprising at least eight contiguous nucleotides having sufficient complementarity to the DRM-encoding nucleic acid of this invention to selectively hybridize with the DRM-encoding nucleic acid of this invention under stringent conditions as described herein and which does not hybridize with nucleic acids which do not encode DRM, under stringent conditions.

“Stringent conditions” refers to the hybridization conditions used in a hybridization protocol or in the primer/template hybridization in a polymerase chain reaction (PCR) protocol. In general, these conditions should be a combination of temperature and salt concentration for hybridizing and washing chosen so that the denaturation temperature is approximately 5-20° C. below the calculated Tₘ (melting/denaturation temperature) of the hybrid under study. The temperature and salt conditions are readily determined empirically in routine, preliminary experiments in which samples of reference nucleic acid are hybridized to the primer nucleic acid of interest and then amplified under conditions of different stringencies. The stringency conditions are readily tested and the parameters altered are readily apparent to one skilled in the art. For example, MgCl₂ concentrations used in PCR buffer can be altered to increase the specificity with which the primer binds to the template, but the concentration range of this compound used in hybridization reactions is narrow and therefore, the proper stringency level is easily determined. For example, hybridizations with oligonucleotide probes which are 18 nucleotides in length can be done at 5-10° C. below the estimated Tₘ in 6× SSPE, then washed at the same temperature in 2× SSPE (62). The Tₘ of such an oligonucleotide can be estimated by allowing 2° C. for each A or T nucleotide and 4° C. for each G or C. An 18 nucleotide probe of 50% G+C would, therefore, have an approximate Tₘ of 54° C. Likewise, the starting salt concentration of an 18 nucleotide primer or probe would be about 100-200 mM. Thus, stringent conditions for such an 18 nucleotide primer or probe would be a Tₘ of about 54° C. and a starting salt concentration of about 150 mM and would be modified accordingly by routine, preliminary experiments. Tₘ values can also be calculated for a variety of conditions utilizing commercially available computer software (e.g., OLIGO®).

Modifications to the nucleic acids of the invention are also contemplated, provided that the essential structure and function of the polypeptide encoded by the nucleic acids is maintained. Likewise, fragments used as primers can have substitutions, provided that a sufficient number of complementary bases exist to allow for selective amplification, as would be determined by routine experimentation (64). In addition, nucleic acid fragments used as probes can have substitutions, provided that enough complementary bases exist to allow for hybridization with the reference sequence to be distinguished from hybridization with other sequences, as would be determined by routine experimentation.

The nucleic acids of this invention can be used as probes, for example, to screen genomic or cDNA libraries or to identify complementary sequences by Northern and Southern blotting. The nucleic acids of this invention can also be used a primers, for example, to transcribe cDNA from RNA and to amplify DNA according to standard amplification protocols, such as PCR, which are well known in the art.

Thus, the present invention further provides a method of detecting and/or quantitating the expression of a nucleic acid encoding the DRM protein in cells in a biological sample by detecting and/or quantitating DNA and/or mRNA which encodes the DRM protein in the cells comprising the steps of: contacting the cells with a detectably labeled nucleic acid probe that hybridizes, under stringent conditions, with DNA and/or mRNA encoding the DRM protein and detecting and/or quantitating the DNA and/or mRNA hybridized with the probe. The mRNA of the cells in the biological sample can be contacted with the probe and detected and/or quantitated according to protocols standard in the art for detecting and quantitating mRNA, including, but not limited to, Northern blotting, dot blotting, ELISPOT assay and PCR amplification. The DNA of the cells in the biological sample can contacted with the probe and detected and/or quantitated according to protocols standard in the art for detecting and quantitating DNA, including, but not limited to, Southern blotting, dot blotting, ELISPOT assay and PCR amplification. The detection and/or quantitation of DNA or mRNA encoding DRM can be used to identify cells which are undergoing, or about to undergo hyperproliferation (i.e., cells which are cancerous or pre-cancerous), as described further below.

The nucleic acid encoding the polypeptide DRM of this invention can be part of a recombinant nucleic acid comprising any combination of restriction sites and/or functional elements as are well known in the art which facilitate molecular cloning and other recombinant DNA manipulations. Thus, the present invention further provides a recombinant nucleic acid comprising the nucleic acid encoding the DRM protein of the present invention. In particular, the isolated nucleic acid encoding DRM and/or a recombinant nucleic acid comprising a nucleic acid encoding DRM can be present in a vector and the vector can be present in a cell, which can be an in vivo cell, an ex vivo cell, a cell cultured in vitro or a cell in a transgenic non-human animal.

Thus, the present invention further provides a vector comprising a nucleic acid encoding DRM. The composition can be in a pharmaceutically acceptable carrier. The vector can be an expression vector which contains all of the genetic components required for expression of the nucleic acid encoding DRM in cells into which the vector has been introduced, as are well known in the art. The expression vector can be a commercial expression vector or it can be constructed in the laboratory according to standard molecular biology protocols. The expression vector can comprise viral nucleic acid including, but not limited to, adenovirus, retrovirus and/or adeno-associated virus nucleic acid. The nucleic acid or vector of this invention can also be in a liposome or a delivery vehicle which can be taken up by a cell via receptor-mediated or other type of endocytosis.

The present invention further provides a method of producing the polypeptide DRM, comprising culturing the cells of the present invention which contain a nucleic acid encoding the polypeptide DRM under conditions whereby the polypeptide DRM is produced. Conditions whereby the polypeptide DRM is produced can include the standard conditions of any expression system, either in vitro or in vivo, in which the polypeptides of this invention are produced in functional form. For example, protocols describing the conditions whereby nucleic acids encoding the DRM proteins of this invention are expressed are provided in the Examples section herein. The polypeptide DRM can be isolated and purified from the cells according to methods standard in the art.

With regard to the polypeptides of this invention, as used herein, “isolated” and/or “purified” means a polypeptide which is substantially free from the naturally occurring materials with which the polypeptide is normally associated in nature. Also as used herein, “polypeptide” refers to a molecule comprised of amino acids which correspond to those encoded by a nucleic acid. The polypeptides of this invention can consist of the entire amino acid sequence of the DRM protein or fragments thereof. The polypeptides or fragments thereof of the present invention can be obtained by isolation and purification of the polypeptides from cells where they are produced naturally or by expression of exogenous nucleic acid encoding the DRM polypeptide. Fragments of the DRM polypeptide can be obtained by chemical synthesis of peptides, by proteolytic cleavage of the polypeptide and by synthesis from nucleic acid encoding the portion of interest. For example, fragments of the DRM polypeptide can comprise the amino acid sequence encoded by nucleotides 4689 through 5147 of SEQ ID NO:5; nucleotides 1339 through 1815 of SEQ ID NO:6; nucleotides 4683 through 5129 of SEQ ID NO:7; nucleotides 4683 through 5033 of SEQ ID NO:8; and nucleotides 4683-5033 of SEQ ID NO:9. The polypeptide may include conservative substitutions where a naturally occurring amino acid is replaced by one having similar properties. Such conservative substitutions do not alter the function of the polypeptide (63).

Thus, it is understood that, where desired, modifications and changes may be made in the nucleic acid and/or amino acid sequence of the DRM polypeptides of the present invention and still obtain a protein having like or otherwise desirable characteristics. Such changes may occur in natural isolates or may be synthetically introduced using site-specific mutagenesis, the procedures for which, such as mis-match polymerase chain reaction (PCR), are well known in the art.

For example, certain amino acids may be substituted for other amino acids in a DRM polypeptide without appreciable loss of functional activity. Since it is the interactive capacity and nature of a protein that defines that protein's biological functional activity, certain amino acid sequence substitutions can be made in a DRM amino acid sequence (or, of course, the underlying nucleic acid sequence) and nevertheless obtain a DRM polypeptide with like properties. It is thus contemplated that various changes may be made in the amino acid sequence of the DRM polypeptide (or underlying nucleic acid sequence) without appreciable loss of biological utility or activity and possibly with an increase in such utility or activity.

The present invention further provides antibodies which specifically bind the DRM polypeptide. The antibodies of the present invention include both polyclonal and monoclonal antibodies. Such antibodies may be murine, fully human, chimeric or humanized. These antibodies can also include Fab or F(ab′)₂ fragments, as well as single chain antibodies (ScFv) (90). The antibodies can be of any isotype IgG, IgA, IgD, IgE and IgM. The antibodies can be produced against peptides which are identified to be immunogenic peptides as described in the Examples provided herein and according to methods well known in the art for identifying immunogenic regions in an amino acid sequence. Such antibodies can be produced by techniques well known in the art which include those described in Kohler et al. (42) or U.S. Pat. Nos. 5,545,806, 5,569,825 and 5,625,126, incorporated herein by reference.

The antibodies of this invention can be used to detect and/or quantitate DRM in a sample. For example, a method is provided for detecting and/or quantitating a DRM protein or antigen in a sample, which can be a biological sample, comprising contacting the sample with an antibody which specifically binds DRM under conditions whereby an antigen/antibody complex can form and detecting the presence of the complex, whereby the presence of the antigen/antibody complex indicates the presence of a DRM protein or antigen in the sample. The amount of the DRM protein in the detected antigen/antibody complex can be determined by methods well known in the art for quantitating protein.

Conditions whereby an antigen/antibody complex can form as well as assays for the detection of the formation of an antigen/antibody complex and quantitating of the detected protein are standard in the art. Such assays can include, but are limited to, Western blotting, immunoprecipitation, immunofluorescence, immunocytochemistry, immunohistochemistry, fluorescence activated cell sorting (FACS), immunomagnetic assays, ELISA, agglutination assays, flocculation assays, cell panning, etc., as are well known to the artisan.

The DRM protein of the present invention has been identified to play a role in regulating a cell's proliferation cycle, as set forth in the Examples provided herein. Thus, the DRM protein of this invention and nucleic acids encoding DRM have therapeutic utility in applications in which it is desirable to alter or control a cell's proliferation cycle.

In particular, the present invention provides a method of arresting cell growth, comprising administering to the cell an effective amount of DRM protein or active fragment thereof. The cell can be in vivo or ex vivo and the DRM protein or active fragment thereof can be in a pharmaceutically acceptable carrier. As used herein, an “active fragment thereof” is a fragment of DRM identified to possess the cell growth arresting activity of the complete protein. Such an active fragment can be identified by producing fragments of the DRM proteins according to standard protocols and assaying the fragments for cell growth arresting activity according to the methods described herein. Also as used herein, “arresting cell growth” means treating or modifying the cell such that the cell is unable to proliferate or form colonies when plated on tissue culture dishes in appropriate media under conditions where similar untreated or unmodified cells, but otherwise identical cells will do so. An effective amount of DRM or active fragment thereof is that amount which results in arrest of cell growth as measured by labeling index, presence of mitotic figures or any other cell proliferation assay now known or developed in the future.

Furthermore, the present invention provides a method of treating or preventing a hyperproliferative cell disorder in a subject diagnosed with, or at risk of developing, a hyperproliferative cell disorder, comprising administering to the subject an effective amount of DRM protein or an active fragment thereof, in a pharmaceutically acceptable carrier. As used herein, an “active fragment thereof” is a fragment of DRM identified to possess the hyperproliferative cell disorder treating or preventing activity of the complete protein. Such an active fragment can be identified by producing fragments of the DRM proteins according to standard protocols and assaying the fragments for hyperproliferative cell disorder treating or preventing activity according to the methods described herein.

The subject can be any animal in which DRM can function in regulating the growth of a cell and can treat or prevent a hyperproliferative cell disorder. For example, the subject can be a mammal and is most preferably a human. As used herein, a “hyperproliferative cell disorder” is any disorder of a cell characterized by unregulated cell division and growth and which has a deleterious effect. An example of a hyperproliferative cell disorder is cancer. Thus, the DRM protein or active fragment thereof of the present invention can be administered to a subject diagnosed with a cancer, to treat the subject's cancer. Examples of cancers include, but are not limited to, leukemia, lymphoma, myeloma, melanoma, sarcoma, bone cancer, prostate cancer, lung cancer, renal cancer, etc.

As stated above, the DRM protein of the present invention can be in a pharmaceutically acceptable carrier and in addition, can include other medicinal agents, pharmaceutical agents, carriers, adjuvants, diluents, immunostimulatory cytokines, etc. By “pharmaceutically acceptable” is meant a material that is not biologically or otherwise undesirable, i.e., the material may be administered to an individual along with the DRM protein without causing substantial deleterious biological effects or interacting in a deleterious manner with any of the other components of the composition in which it is contained. Actual methods of preparing such dosage forms are known, or will be apparent, to those skilled in this art; for example, see Remington's Pharmaceutical Sciences (91).

To determine the effect of the administration of the DRM polypeptide or active fragment thereof on inhibition of tumor cell growth in laboratory animals, the animals can either be pre-treated with the DRM polypeptide or active fragment thereof and then challenged with a lethal dose of tumor cells, or the lethal dose of tumor cells can be administered to the animal prior to receipt of the DRM polypeptide or active fragment thereof and survival times documented. To determine the amount of DRM polypeptide or active fragment thereof which would be an effective tumor cell growth-inhibiting amount, animals can be treated with tumor cells as described herein and varying amounts of the DRM polypeptide or active fragment thereof can be administered to the animals. Standard clinical parameters, as described herein, can be measured and that amount of DRM polypeptide or active fragment thereof effective in inhibiting tumor cell growth can be determined. These parameters, as would be known to one of ordinary skill in the art of oncology and tumor biology, can include, but are not limited to, physical examination of the subject, measurements of tumor size, measurements of levels of circulating tumor antigen, X-ray studies and biopsies, as well as any other assay now known or later identified as a diagnostic and/or prognostic assay for tumor cell growth.

In vitro assays can also be utilized to determine the effect of the administration of the DRM polypeptide or active fragment thereof on inhibition of tumor cell growth. These assays are well known in the art and include in vitro invasiveness assays.

Once dosages effective in treating hyperproliferative cell disorders, such as cancer, are determined for animal models, these data can be extrapolated to determine approximate effective treatment dosages in humans (e.g., by correlating mg/kg body weight of an amount of DRM protein effective in animals). Specific effective hyperproliferative cell disorder treating dosages in humans can be determined according to standard protocols established for clinical trials, as are well documented in the art (45-49). To determine the efficacy of administration of a given dose of the DRM polypeptide or active fragment thereof for treating hyperproliferative cell disorders, such as cancer, in humans, standard clinical response parameters can be analyzed, as described herein and as are well known in the art.

Additionally, the efficacy of administration of a particular dose of DRM protein or active fragment thereof in preventing a hyperproliferative cell disorder, such as cancer, in a subject not known to have a hyperproliferative cell disorder, but known to be at risk of developing a hyperproliferative cell disorder, can be determined by evaluating standard signs, symptoms and objective laboratory tests, known to one of skill in the art, over time after administration of the DRM polypeptide or active fragment thereof. This time interval may be short (weeks/months) or long (years/decades). The determination of who would be at risk for the development of a hyperproliferative cell disorder would be made based on current knowledge of the known risk factors for a particular disorder familiar to clinicians and researchers in this field, such as a particularly strong family history of a disorder. Furthermore, a subject can be identified as being at risk of developing a hyperproliferative disorder, such as cancer, according to the methods provided herein.

The DRM polypeptide or active fragment thereof of this invention can be administered to the subject orally or parenterally, as for example, by intramuscular injection, by intraperitoneal injection, topically, transdermally, injection directly into the tumor, or the like, although subcutaneous injection is typically preferred. Tumor cell growth inhibiting and cancer treating amounts of the DRM polypeptide or active fragment thereof can be determined using standard procedures, as described. The exact dosage of the DRM polypeptide or active fragment thereof will vary from subject to subject, depending on the species, age, weight and general condition of the subject, the severity of the cancer or disorder that is being treated, the mode of administration and the like. Thus, it is not possible to specify an exact amount. However, an appropriate amount may be determined by one of ordinary skill in the art using only routine screening given the teachings herein.

For oral administration, fine powders or granules may contain diluting, dispersing, and/or surface active agents and may be presented in water or in a syrup, in capsules or sachets in the dry state, or in a nonaqueous solution or suspension wherein suspending agents may be included, in tablets wherein binders and lubricants may be included, or in a suspension in water or a syrup. Where desirable or necessary, flavoring, preserving, suspending, thickening, or emulsifying agents may be included. Tablets and granules are preferred oral administration forms and these may be coated.

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system, such that a constant dosage level is maintained. See, e.g., U.S. Pat. No. 3,710,795, which is incorporated by reference herein.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc. an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art (91).

Generally, to treat or prevent a hyperproliferative cell disorder in a subject, the dosage of DRM protein or active fragment thereof will approximate that which is typical for the administration of proteins and typically, the dosage will be in the range of about 1 to 500 μg of the DRM polypeptide or active fragment thereof per dose, and preferably in the range of 50 to 250 μg of the DRM polypeptide or active fragment thereof per dose. This amount can be administered to the subject once every other week for about eight weeks or once every other month for about six months. The effects of the administration of the DRM polypeptide or active fragment thereof can be determined starting within the first month following the initial administration and continued thereafter at regular intervals, as needed, for an indefinite period of time.

As described herein, the present invention also provides a nucleic acid and a vector, which can be in a pharmaceutically acceptable carrier, which encodes the DRM polypeptide or active fragments thereof, of the present invention. Such nucleic acids can be used in gene therapy protocols to treat or prevent hyperproliferative cell disorders, such as a cancer, in a subject.

Thus, the present invention further provides a method of treating a hyperproliferative cell disorder in a subject diagnosed with a hyperproliferative cell disorder, comprising administering an effective amount of the nucleic acid of this invention, which encodes the DRM protein or an active fragment thereof, to a cell of the subject under conditions whereby the nucleic acid is expressed in the subject's cell, thereby treating the hyperproliferative cell disorder.

Also provided is a method of arresting the growth of a cell, comprising administering to the cell an effective amount of a nucleic acid encoding a DRM protein or an active fragment thereof, to a cell under conditions whereby the nucleic acid is expressed in the cell, thereby arresting the growth of the cell.

The present invention further provides a method of inhibiting tumor cell growth, comprising administering to a tumor cell an effective amount of a nucleic acid encoding a DRM protein or an active fragment thereof, to a tumor cell under conditions whereby the nucleic acid is expressed in the tumor cell, thereby inhibiting tumor cell growth.

The nucleic acid can be administered to the cell in a virus, which can be, for example, adenovirus, retrovirus and adeno-associated virus. Alternatively, the nucleic acid of this invention can be administered to the cell as naked DNA or in a liposome. The cell can be either in vivo or ex vivo. Also, the cell can be any cell which can take up and express exogenous nucleic acid and produce the DRM polypeptide or fragment thereof of this invention.

If ex vivo methods are employed, cells or tissues can be removed and maintained outside the subject's body according to standard protocols well known in the art. The nucleic acids of this invention can be introduced into the cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or transplanted back into the subject per standard methods for the cell or tissue type. Methods for transplantation or infusion of various cells into a subject are well known in the art.

For in vivo methods, the nucleic acid encoding the DRM protein or active fragments thereof, can be administered to the subject in a pharmaceutically acceptable carrier as further described herein.

In the methods described above which include the administration and uptake of exogenous nucleic acid into the cells of a subject (i.e., gene transduction or transfection), the nucleic acids of the present invention can be in the form of naked nucleic acid or the nucleic acids can be in a vector for delivering the nucleic acids to the cells for expression of the DRM protein or active fragment thereof. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g.,50,51). The recombinant retrovirus can then be used to infect and thereby deliver to the infected cells nucleic acid encoding the DRM protein. The exact method of introducing the exogenous nucleic acid into mammalian cells is, of course, not limited to the use of retroviral vectors. Other techniques are widely available for this procedure including the use of adenoviral vectors (52), adeno-associated viral (AAV) vectors (53), lentiviral vectors (54), pseudotyped retroviral vectors (55). Physical transduction techniques can also be used, such as liposome delivery and receptor-mediated and other endocytosis mechanisms (see, for example, 56). This invention can be used in conjunction with any of these or other commonly used gene transfer methods.

Various adenoviruses may be used in the compositions and methods described herein. For example, a nucleic acid encoding the DRM protein can be inserted within the genome of adenovirus type 5. Similarly, other types of adenovirus may be used such as type 1, type 2, etc. For an exemplary list of the adenoviruses known to be able to infect human cells and which therefore can be used in the present invention, see Fields, et al. (57). Furthermore, it is contemplated that a recombinant nucleic acid comprising an adenoviral nucleic acid from one type adenovirus can be packaged using capsid proteins from a different type adenovirus.

The adenovirus of the present invention is preferably rendered replication deficient, depending upon the specific application of the compounds and methods described herein. Methods of rendering an adenovirus replication deficient are well known in the art. For example, mutations such as point mutations, deletions, insertions and combinations thereof, can be directed toward a specific adenoviral gene or genes, such as the E1 gene. For a specific example of the generation of a replication deficient adenovirus for use in gene therapy, see WO 94/28938 (Adenovirus Vectors for Gene Therapy Sponsorship) which is incorporated herein.

In the present invention, the nucleic acid encoding the DRM protein or active fragment thereof (DRM-encoding insert) can be inserted within an adenoviral genome and the DRM-encoding insert can be positioned such that an adenovirus promoter is operatively linked to the DRM-encoding insert such that the adenoviral promoter can then direct transcription of the nucleic acid, or the DRM-encoding insert may contain its own adenoviral promoter. Similarly, the DRM-encoding insert may be positioned such that the nucleic acid encoding the DRM protein or fragment may use other adenoviral regulatory regions or sites such as splice junctions and polyadenylation signals and/or sites. Alternatively, the nucleic acid encoding the DRM protein or fragment may contain a different enhancer/promoter (e.g., CMV or RSV-LTR enhancer/promoter sequences) or other regulatory sequences, such as splice sites and polyadenylation sequences, such that the nucleic acid encoding the DRM protein or fragment may contain those sequences necessary for expression of the DRM protein fragment and not partially or totally require these regulatory regions and/or sites of the adenovirus genome. These regulatory sites may also be derived from another source, such as a virus other than adenovirus. For example, a polyadenylation signal from SV40 or BGH may be used rather than an adenovirus, a human, or a murine polyadenylation signal. The DRM-encoding insert may, alternatively, contain some sequences necessary for expression of the nucleic acid encoding the DRM protein or fragment and derive other sequences necessary for the expression of the DRM-encoding insert from the adenovirus genome, or even from the host in which the recombinant adenovirus is introduced.

As another example, for administration of nucleic acid encoding the DRM protein or active fragment thereof to an individual in an AAV vector, the AAV particle can be directly injected intravenously. The AAV has a broad host range, so the vector can be used to transduce any of several cell types, but preferably cells in those organs that are well perfused with blood vessels. To more specifically administer the vector, the AAV particle can be directly injected into a target organ, such as muscle, liver or kidney. Furthermore, the vector can be administered intraarterially, directly into a body cavity, such as intraperitoneally, or directly into the central nervous system (CNS).

An AAV vector can also be administered in gene therapy procedures in various other formulations in which the vector plasmid is administered after incorporation into other delivery systems such as liposomes or systems designed to target cells by receptor-mediated or other endocytosis procedures. The AAV vector can also be incorporated into an adenovirus, retrovirus or other virus which can be used as the delivery vehicle.

As described above, the nucleic acid or vector of the present invention can be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The mode of administration of the nucleic acid or vector of the present invention can vary predictably according to the disorder being treated and the tissue being targeted. For example, for administration of the nucleic acid or vector in a liposome, catheterization of an artery upstream from the target organ is a preferred mode of delivery, because it avoids significant clearance of the liposome by the lung and liver.

The nucleic acid or vector may be administered orally, parenterally (e.g., intravenously), by intramuscular injection, by intraperitoneal injection, transdermally, extracorporeally, topically or the like, although intravenous administration is typically preferred. The exact amount of the nucleic acid or vector required will vary from subject to subject, depending on the species, age, weight and general condition of the subject, the severity of the disorder being treated, the particular nucleic acid or vector used, its mode of administration and the like. Thus, it is not possible to specify an exact amount for every nucleic acid or vector. However, an appropriate amount can be determined by one of ordinary skill in the art using only routine experimentation given the teachings herein (see, e.g., Remington 's Pharmaceutical Sciences).

As one example, if the nucleic acid of this invention is delivered to the cells of a subject in an adenovirus vector, the dosage for administration of adenovirus to humans can range from about 10⁷ to 10⁹ plaque forming units (pfu) per injection, but can be as high as 10¹² pfu per injection (59,60). Ideally, a subject will receive a single injection. If additional injections are necessary, they can be repeated at six month intervals for an indefinite period and/or until the efficacy of the treatment has been established.

Parenteral administration of the nucleic acid or vector of the present invention, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

To determine the effect of the administration of the nucleic acid of this invention on inhibition of tumor cell growth in laboratory animals, the animals can either be pre-treated with the nucleic acid and then challenged with a lethal dose of tumor cells, or the lethal dose of tumor cells can be administered to the animal prior to receipt of the nucleic acid and survival times documented. To determine the amount of nucleic acid which would be an effective tumor cell growth-inhibiting amount, animals can be treated with tumor cells as described herein and varying amounts of the nucleic acid can be administered to the animals. Standard clinical parameters, as described herein, can be measured and the amount of DRM encoding nucleic acid effective in inhibiting tumor cell growth can be determined. These parameters, as would be known to one of ordinary skill in the art of oncology and tumor biology, can include, but are not limited to, physical examination of the subject, measurements of tumor size, measurements of levels of circulating tumor antigen, X-ray studies and biopsies, as well as any other assay now known or later identified as a diagnostic and/or prognostic assay for tumor cell growth.

Once dosages effective in inhibiting cell growth and/or treating hyperproliferative cell disorders, such as cancer, are determined for animal models, these data can be extrapolated to determine approximate effective treatment dosages in humans. Specific effective hyperproliferative cell disorder treating dosages of DRM-encoding DNA in humans can be determined according to standard protocols established for clinical trials, as are well documented in the art. To determine the efficacy of administration of a given dose of the DRM-encoding nucleic acid for treating hyperproliferative cell disorders, such as cancer, in humans, standard clinical response parameters can be analyzed, as described herein and as are well known in the art.

Additionally, the efficacy of administration of a particular dose of DRM encoding nucleic acid in preventing a hyperproliferative cell disorder, such as cancer, in a subject not known to have a hyperproliferative cell disorder, but known to be at risk of developing a hyperproliferative cell disorder, can be determined by evaluating standard signs, symptoms and objective laboratory tests, known to one of skill in the art, over time after administration of the DRM encoding nucleic acid. This time interval may be short (weeks/months) or long (years/decades). The determination of who would be at risk for the development of a hyperproliferative cell disorder would be made based on current knowledge of the known risk factors for a particular disorder familiar to clinicians and researchers in this field, such as a particularly strong family history of a disorder. Furthermore, a subject can be identified as being at risk of developing a hyperproliferative disorder, such as cancer, according to the methods provided herein.

As described herein, the DRM protein is produced in normal cells (i.e., cells which are differentiating normally) at detectable levels. Tumor cells and cells which have been transformed by transfection with an oncogene do not produce detectable levels of DRM protein. A decrease in the level of DRM protein or RNA, or such a decrease in a particular differentiating lineage which normally expresses DRM during differentiation, can be diagnostic of a premalignant or early malignant state. Thus, the present invention provides a method for the early identification of malignancies or premalignant states.

Thus, further provided in the present invention is a method of identifying a subject at risk of developing a hyperproliferative cell disorder (e.g., cancer), comprising measuring the amount of DRM protein or the amount of nucleic acid encoding DRM in a cell of the subject, whereby an amount of DRM protein or nucleic acid encoding DRM in a cell less than the amount of DRM protein or nucleic acid encoding DRM in a cell of a normal subject identifies a subject at risk of developing a hyperproliferative cell disorder. The cell of the subject is a cell which produces DRM and can be, but is not limited to cells of the brain, lung, intestine and esophagus (goblet cells), as well as any other cell now known or later identified to produce DRM.

The amount of DRM protein in a cell can be determined by methods standard in the art for quantitating proteins in a cell, such as Western blotting, ELISA, ELISPOT, immunoprecipitation, immunofluorescence (e.g., FACS), immunohistochemistry, immunocytochemistry, etc., as well as any other method now known or later developed for quantitating protein in a cell.

The amount of nucleic acid encoding DRM in a cell can be determined by methods standard in the art for quantitating nucleic acid in a cell, such as in situ hybridization, quantitative PCR, Northern blotting, ELISPOT, dot blotting, etc., as well as any other method now known or later developed for quantitating nucleic acid in a cell.

The cell can be a separate cell or a cell in intact tissue, which can be a biopsy specimen. As used herein, “a cell of a normal subject” means a cell or tissue which is histologically normal and was obtained from a subject believed to be without malignancy and having no increased rick of developing a malignancy or was obtained from tissues adjacent to tissue known to be malignant and which is determined to be histologically normal (non-malignant) as determined by a pathologist.

The present invention is further based on the unexpected discovery that fusion of DRM or active fragments thereof, with enhanced green fluorescent protein (EGFP) or active fragments thereof, yields a protein which is localized to the nucleus, rather than the cytoplasm, and results in an improved EGFP which has greater stability than conventional EGFP, providing a much more versatile research tool for use in screening assays, protein-protein interaction studies and cell marking applications.

Thus, the present invention provides a fusion polypeptide comprising a DRM protein region and a green fluorescent protein region. For example, the fusion polypeptide of this invention can be a polypeptide having the amino acid sequence of SEQ ID NO:29. The fusion polypeptide of this invention can comprise the entire DRM protein or an active fragment thereof and the entire EGFP or an active fragment thereof. The identification of an active fragment of either DRM or EGFP can be carried out according to routine methods for identifying active fragments. For example, a fragment of either protein can be produced by PCR amplification of a specific region of the protein, by deleting portions of the protein at specific restriction sites with restriction endonucleases, by introducing stop codons into the protein sequence, by synthesizing a peptide comprising a fragment of the protein, etc., as would be well known to one of skill in the art. The resulting fragments can be tested for functional activity according to the methods provided herein as well as are described in the art. For example, the fusion protein of this invention can have the amino acid sequence of SEQ ID NOS:30, 31, 32, 33, 34 and 35, encoded by the nucleic acids of SEQ ID NOS:5, 6, 7, 8, 9 and 19, respectively. The production of each of the fusion proteins having the amino acid sequences of SEQ ID NOS:30-35 is described in the Examples section herein.

The present invention further provides a green fluorescent protein having increased stability, comprising a fusion protein comprising a DRM protein amino acid sequence linked to an EGFP amino acid sequence. As used herein, “having increased stability” means that the EGFP of the EGFP/DRM fusion protein maintains fluorescence activity when exposed to fixatives (e.g., ethanol, methanol, acetone), detergents (e.g., TritonX100, NP40), or other conditions under which the fluorescence activity of unfused (conventional) EGFP is greatly diminished (>75%) or no longer detectable.

An isolated nucleic acid encoding the fusion polypeptides described above is also provided. The isolated nucleic acid of this invention which encodes the EGFP/DRM fusion protein can be a nucleic acid having the nucleotide sequence of SEQ ID NO: 1. By “isolated nucleic acid” is meant a nucleic acid molecule that is substantially free of the other nucleic acids and other components commonly found in association with nucleic acid in a cellular environment. Separation techniques for isolating nucleic acids from cells are well known in the art and include phenol extraction followed by ethanol precipitation and rapid solubilization of cells by organic solvent or detergents (35).

The nucleic acid encoding the fusion polypeptide can be any nucleic acid that functionally encodes the fusion polypeptide. To functionally encode the polypeptide (i.e., allow the nucleic acid to be expressed), the nucleic acid can include, for example, expression control sequences, such as an origin of replication, a promoter, an enhancer and necessary information processing sites, such as ribosome binding sites, RNA splice sites, polyadenylation sites and transcriptional terminator sequences. Preferred expression control sequences are promoters derived from metallothionine genes, actin genes, immunoglobulin genes, CMV, SV40, adenovirus, bovine papilloma virus, etc. A nucleic acid encoding a selected fusion polypeptide can readily be determined based upon the genetic code for the amino acid sequence of the selected fusion polypeptide and many nucleic acids will encode any selected fusion polypeptide. Modifications in the nucleic acid sequence encoding the fusion polypeptide are also contemplated. Modifications that can be useful are modifications to the sequences controlling expression of the fusion polypeptide to make production of the fusion polypeptide inducible or repressible as controlled by the appropriate inducer or repressor. Such means are standard in the art (35). The nucleic acids can be generated by means standard in the art, such as by recombinant nucleic acid techniques, as exemplified in the examples herein and by synthetic nucleic acid synthesis or in vitro enzymatic synthesis.

A vector comprising the nucleic acids encoding the fusion proteins of the present invention and a cell comprising the vector are also provided. The vector can be in a host (e.g., cell line or transgenic animal) that can express the fusion polypeptide contemplated by the present invention.

There are numerous E. coli (Escherichia coli) expression systems known to one of ordinary skill in the art useful for the expression of nucleic acid encoding proteins such as fusion proteins. Other microbial hosts suitable for use include bacilli, such as Bacillus subtilis, and other enterobacteria, such as Salmonella and Serratia, as well as various Pseudomonas species. These prokaryotic hosts can support expression vectors which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence and have ribosome binding site sequences for example, for initiating and completing transcription and translation. If necessary, an amino terminal methionine can be provided by insertion of a Met codon 5′ and in-frame with the protein sequences. Also, the carboxy-terminal extension of the protein can be removed using standard oligonucleotide mutagenesis procedures.

Additionally, yeast expression can be used. There are several advantages to yeast expression systems. First, evidence exists that proteins produced in a yeast secretion system exhibit correct disulfide pairing. Second, post-translational glycosylation is efficiently carried out by yeast secretory systems. The Saccharomyces cerevisiae pre-pro-alpha-factor leader region (encoded by the MFα-1 gene) is routinely used to direct protein secretion from yeast (89). The leader region of pre-pro-alpha-factor contains a signal peptide and a pro-segment which includes a recognition sequence for a yeast protease encoded by the KEX2 gene. This enzyme cleaves the precursor protein on the carboxyl side of a Lys-Arg dipeptide cleavage-signal sequence. The polypeptide coding sequence can be fused in-frame to the pre-pro-alpha-factor leader region. This construct is then put under the control of a strong transcription promoter, such as the alcohol dehydrogenase I promoter or a glycolytic promoter. The protein coding sequence is followed by a translation termination codon, which is followed by transcription termination signals. Alternatively, the polypeptide coding sequence of interest can be fused to a second protein coding sequence, such as Sj26 or β-galactosidase, used to facilitate purification of the fusion protein by affinity chromatography. The insertion of protease cleavage sites to separate the components of the fusion protein is applicable to constructs used for expression in yeast.

Efficient post-translational glycosylation and expression of recombinant proteins can also be achieved in Baculovirus systems in insect cells.

Mammalian cells permit the expression of proteins in an environment that favors important post-translational modifications such as folding and cysteine pairing, addition of complex carbohydrate structures and secretion of active protein. Vectors useful for the expression of proteins in mammalian cells are characterized by insertion of the protein coding sequence between a strong viral promoter and a polyadenylation signal. The vectors can contain genes conferring either gentamicin or methotrexate resistance for use as selectable markers. The fusion protein coding sequence can be introduced into a Chinese hamster ovary (CHO) cell line using a methotrexate resistance-encoding vector. Presence of the vector RNA in transformed cells can be confirmed by Northern blot analysis and production of a cDNA or opposite strand RNA corresponding to the fusion protein coding sequence can be confirmed by Southern and Northern blot analysis, respectively. A number of other suitable host cell lines capable of secreting intact proteins have been developed in the art and include the CHO cell lines, HeLa cells, myeloma cell lines, Jurkat cells and the like. Expression vectors for these cells can include expression control sequences, as described above.

The vectors containing the nucleic acid sequences of interest can be transferred into the host cell by well-known methods, which vary depending on the type of cell host. For example, calcium chloride transfection is commonly utilized for prokaryotic cells, whereas calcium phosphate treatment or electroporation may be used for other cell hosts.

Alternative vectors for the expression of protein in mammalian cells, similar to those developed for the expression of human gamma-interferon, tissue plasminogen activator, clotting Factor VIII, hepatitis B virus surface antigen, protease Nexinl, and eosinophil major basic protein, can be employed. Further, the vector can include CMV promoter sequences and a polyadenylation signal available for expression of inserted nucleic acid in mammalian cells (such as COS7).

The nucleic acid sequences can be expressed in hosts after the sequences have been positioned to ensure the functioning of an expression control sequence. These expression vectors are typically replicable in the host organisms either as episomes or as an integral part of the host chromosomal DNA. Commonly, expression vectors can contain selection markers, e.g., tetracycline resistance or hygromycin resistance, to permit detection and/or selection of those cells transformed with the desired nucleic acid sequences (see, e.g., U.S. Pat. No. 4,704,362).

Thus, further provided is a method of producing the green fluorescent protein having increased stability of this invention, comprising the steps of producing a nucleic acid construct whereby a first nucleic acid sequence encoding EGFP or an active fragment thereof is positioned upstream and in frame with a second nucleic acid encoding DRM or an active fragment thereof; cloning the nucleic acid construct into an expression vector; and placing the expression vector into a cell under conditions whereby the nucleic acid of the construct will be expressed, thereby producing a green fluorescent protein having increased stability. The expression vector and expression system can be of any of the types as described herein. The cloning of the first and second nucleic acids into the expression vector and expression of the nucleic acids under conditions which allow for the production of the fusion protein of this invention can be carried out as described in the Examples section included herein. The method of this invention can further comprise the step of isolating and purifying the fusion polypeptide, according to methods well known in the art and as described herein.

The EGFP/DRM fusion protein of this invention improves the stability of the EGFP as compared to conventional EGFP. Thus, the fusion protein of this invention can be used in assays for which conventional EGFP is not suitable, such as fluorescence-based assays which require cell fixation and in protocols where cell marking is necessary or desired. For example, the EGFP/DRM fusion protein of this invention can be used in cell cycle analysis using PI or BudR, where fixation is required to allow the dye to enter in to the cell nucleus. Also, the stabilized EGFP of this invention can be introduced as a marker (e.g., linked to a ligand to detect the presence of a receptor) or the nucleic acid encoding the stabilized EGFP can be used to identify cells into which a particular expression construct is introduced or where a reporter gene signal is desired.

The stabilized EGFP of this invention can also be linked to proteins or antibodies for use in ELISA assays. The advantage of using stabilized EGFP is that the stabilized EGFP can be attached as a particular protein is being synthesized, so that materials which could not be chemically modified to attach fluorescent groups because of stability problems could be labeled. The stabilized EGFP can also be used as a marker during purification. For example, materials can be produced in vivo in fermentor-type production facilities and a desired material can be purified by the presence of the EGFP protein marker.

The present invention is more particularly described in the following examples which are intended as illustrative only since numerous modifications and variations therein will be apparent to those skilled in the art.

### EXAMPLES

### Example I

### Isolation and Characterization of Rat drm Gene and Gene Product

Cell culture.

The REF-1, DTM, F-1 and ST33c rat cell lines have previously been described (40-42). DTM and ST33c cell lines were maintained at 34° C. in DMEM with 5% fetal calf serum, while REF-1, as well as REF-1 cells transformed by different oncogenes, were grown at 37° C. in DMEM (Gibco) with 5% or 10% fetal calf serum.

DNA and RNA Analysis.

High molecular weight DNA was purified by standard procedures (15) and analyzed by Southern blotting (35). Total RNA was extracted from culture cells by RNAzolB (Tel-Teck, Inc., Texas) (7), and 10 μg was used per lane in a Northern analysis. Filters were pre-hybridized and hybridized at 42° C. for 18-20 hr in 5× SSPE (NaCl, NaH₂PO₄, Na₂EDTA, pH 7.4) containing 10× Denhardt's solution (9), 2% SDS, 50% formamide, and 100 μg of heat-denatured salmon sperm DNA per ml, the filters were washed sequentially in 2× SSC/0.05% SDS at room temperature for 30 min and in 0.1× SSC/0.1% SDS at 50° C. for 40 min. Autoradiography was for 2-4 days at −70° C. with an intensifying screen. Poly(A)⁺ was isolated by using the “Fast Track” mRNA isolation kit (InVitrogen) according to the manufacturer's specifications. Multi-tissue Northern blot (Clontech) was treated according to the manufacturer's protocol.

The murine recombinant retrovirus expressing v-src was obtained from S. M. Anderson. The vector expressing activated ras is pEJ-ras (38) containing the Val¹²-mutated fragments of human c-ras in pBR322.

Identification and Isolation of drm cDNA.

Messenger RNAs expressed differentially in DTM and F-1 cells were displayed as described by Liang and Pardee (25). First-strand cDNAs were synthesized on 1.5 μg of polyadenylated RNA extracted from either cell line using the “cDNA Cycle Kit for RT-PCR” (Invitrogen) and specific primers T12VA, T12VC (V was either A, C, G). cDNAs were then amplified by polymerase chain reaction (PCR) using [α-³⁵S]dATP and combinations of 3′ specific primers and arbitrary 5′ primers [AGCCAGCGAA (SEQ ID NO:22), GACCGCTTGT (SEQ ID NO:23), AGGTGACCGT (SEQ ID NO:24), GGTACTCCAC (SEQ ID NO:25), GTTGCGATCC (SEQ ID NO:26)]. PCR products were separated on a 6% polyacrylamide gel and visualized by autoradiography.

Screening of cDNA Library.

An oligo dT-primed cDNA library of rat embryo fibroblasts constructed in a λZAP XR vector, was screened with the 691 bp drm cDNA isolated from F-1 mRNA by the differential display technique, as described (35). Three independent clones (C13ZAP, C17ZAP and C110ZAP) were isolated and further analyzed. 5′ sequences of the C17ZAP absent from the other clones were used as probes to screen a rat kidney 5′-stretch λgt11 cDNA library (Clontech). Two clones (C17gt, C110gt) were isolated, further amplified and analyzed. cDNA clones were sequenced on both strands by the dideoxy chain termination method using the “T7 sequencing kit” (Pharmacia Biotech) (36). Portions of the sequencing data were compiled and analyzed by using the University of Wisconsin Genetics Computer Group package (11).

Protein Analysis.

1) In Vitro Transcription and Translation.

The 2.1 kb EcoRI fragment of Clone 10 gt, as well as the BamHI/KpnI fragment from this insert, both containing the putative drm coding region, were inserted into the Bluescript KS vector. Plasmid DNAs were transcribed and translated using the TNT T7 and T3 reticulocyte lysate system (Promega) with L-³⁵S-cysteine (1200 Ci/mmol, Amersham). Translation products were separated by SDS-PAGE and processed for fluorography. T7 polymerase produces a sense message, while T3 produces an antisense product. Luciferase DNA was used as a positive control.

2) Construction of Tagged drm Protein-Expression Vector.

The coding region of drm cDNA was fused in frame at its 3′ end with the DNA fragment encoding the nine residue epitope of the HA-1 influenza virus hemagglutinin by polymerase chain reaction. The primers used were: 5′ (5′-CCGCTCGAGGTGACAGAATGAATCGC-3′) (SEQ ID NO:27) and 3′ (5′CCCGTTAACTTAGGCGTAGTCGGGCACGTCGTAGGGGTAATCCAAGTCG AT3′) (SEQ ID NO:28). The 5′ primer introduces an XhoI restriction site, while the 3′ primer removes the stop codon from the drm and introduces another one downstream from the inserted HA-1 sequence. It also introduces an HpaI site downstream from the stop codon. The PCR product was digested with XhoI/HpaI and inserted into the pSVL expression vector (39) between the XhoI and SmaI sites.

3) Preparation and Characterization of Antibodies.

Two peptides based on the predicted sequence of drin protein were selected to raise rabbit polyclonal antibodies. An N-terminal cysteine residue was added to the first peptide (990), which corresponds to amino acids 79-92 to enable coupling of the peptide to KLH (keyhole limpet hemocyanim) carrier protein prior to immunization. The second peptide (987), corresponding to amino acids 158-172, was coupled to the carrier protein through a natural cysteine residue on its N-terminal end. A peptide which corresponds to amino acids 33-52 was expressed as a fusion with bacteriophage MS2 coat protein and used to immunize rabbits as described herein.

4) Immunoprecipitation and Western Blotting.

Cell lysates prepared under denaturing conditions were either first immunoprecipitated using either drm-specific 990 antisera or anti-HA monoclonal antibody (Babco), followed by separation on SDS-PAGE and Western blotting, or total lysates were analyzed by SDS-PAGE and Western blotting.

For immunoblotting, proteins were electrophoretically transferred to nitrocellulose at 60 mA for 2 hrs. Filters were incubated first with the appropriate primary antibody and then with horseradish peroxidase-labeled secondary antibodies (Amersham). Antibodies were detected using the ECL detection system (Amersham) or the Super Signal CL-HRP Substrate System (Pierce) and visualized using Kodak XAR-5 X-ray film.

Western blots were “stripped” for reprobing with other primary antibodies according to the manufacturer's protocol (Amersham).

Transfection of drm expression vectors. For stable transfection experiments, cDNA containing the full-length drm ORF was inserted into the BamHI and KpnI restriction sites of the pMEXneo expression vector (21). In this construct, drm and the neo-selectable marker were under the control of an MuLV LTR and an SV40 promoter, respectively. For colony formation assays, 5×10⁵ cells were overlaid with a mixture consisting of 5 μg pMEXdrm or expression vector alone and 30 μl DOTAP (Boehringer Mannheim). After 6 hours this mixture was replaced with regular media and the cultures maintained for another 48 hours. Cells were then split 1:3, grown in the presence of G418 (Life Technologies; effective concentration, 400 μg/ml) for 2 weeks and colonies resistant to G418 were counted and isolated. Growth temperatures for transfected cells were: for REF-1 and CHO, 37° C.; for DTM, 34° C.; and for ST33c, 34° C. and 39° C. Transient transfections of Cos-7 cells were performed using the pSVL vector expressing a HA-tagged drm and LipofectAMINE (Life Technologies, Gaithersburg, Md.), according to the manufacturer's specifications.

In Situ Hybridization.

Tissues from Sprague-Dawley rats were processed and analyzed by in situ hybridization according to D. Sassoon (37). A non-radioactive riboprobe containing 1.9 kb of the 3′ end of drm was generated by using Digoxigenin RNA Labeling Kit (SP6/T7) from Boehringer Mannheim, and concentration of the labeled probe was determined by using the SIG Nucleic Acid Detection Kit (Boehringer Mannheim). Detection was performed by using Anti-Digoxigenin antibody, conjugated with Alkaline Phosphatase (Nucleic Acid Detection Kit, Boehringer Mannheim). Sections were counterstained with Methyl Green (1%) and mounted in Aqueous Mounting Medium (Signet Laboratories). Analysis was performed on a Nikon Labophot 2 microscope.

Analysis of Apoptosis.

ST33c cells were transfected with the control vector or with the vector containing drm at 34° C., and pools of G418-resistant colonies were selected, expanded and analyzed for expression of drm-specific mRNA. ST33c cells expressing drm were shifted to 39° C. for 24 hrs, and cells were fixed in 3.7% formaldehyde in PBS (10 min, RT), washed three times, stained in DAPI (10 min, RT) and examined with a Nikon inverted microscope under UW illumination. DNA fragmentation analysis was performed as previously described (1).

Nucleotide Sequence Accession Number.

The drm sequence for the rat homologue has been assigned GenBank/EMBL accession number Y10019.

The characterization of a flat (non-transformed) revertant cell line, F-1, which was isolated from rat fibroblasts (DTM) transformed by the serine/threonine kinase oncogene mos has been previously reported (41). F-1 cells express high levels of v-mos-specific RNA and kinase activity, but fail to express characteristic transformed properties, including colony formation in soft agar and tumor formation in nude mice. Moreover, the revertants are resistant to re-transformation by v-mos and v-raf while they can be efficiently transformed by v-ras and, with a somewhat lower efficiency, v-src. The reversion and resistance to re-transformation correlated with the failure of the serine/threonine kinase oncogenes v-mos and v-raf to activate the MAP kinase pathway due to their inability to activate MEK-1 or MEK-2, the immediate upstream activators of MAP kinase.

Since levels of MEK and MAP kinase were not changed in the revertant cells, and since growth factors and ras activated MEK and the MAP kinase cascade normally, these results suggested that the reversion could be the result of mutations affecting the expression or function of genes which contribute to the activation of MEK by v-mos or v-raf, or from the expression in the revertant cells of genes which block this activation and which are down-regulated in DTM and other transformed cells. In an attempt to identify such transcriptional changes, differential display analysis was used to compare the expression of RNA in transformed and revertant cells. Described herein is the identification and characterization of a novel cDNA, designated drm (down-regulated in v-mos-transformed cells), which is expressed in the F-1 revertant and normal parental rat fibroblasts, but which is down-regulated in rat fibroblasts transformed by several retroviral oncogenes. The drm cDNA shows no significant homologies to known genes in DNA databases and contains an open reading frame (ORF) capable of encoding an 184 amino acid, cysteine-rich protein with a calculated molecular weight of 20,682. Regions of the drm protein show significant sequence homologies with the rat and human DAN (NO3) gene products (10, 28-30), which have been shown to possess tumor and growth-suppressing activities. The drm gene encodes a 20.7 kDa protein recognized by a specific antiserum in phenotypically normal rat cells. This protein was not detected in v-mos-transformed cells. Analysis of RNA from multiple tissues of the rat and in situ hybridization experiments in adult rats, indicate that drm expression is regulated in a tissue-specific manner. In situ analysis also indicate that drm RNA is predominantly expressed in terminally-differentiated, non-dividing cells, such as neurons, type-1 cells of the lung, and goblet cells of the intestine.

Transfection analysis demonstrates that drm overexpression in normal rat fibroblasts blocks cell proliferation, while co-transfection with ras oncogene reverses this inhibition. Furthermore, cells overexpressing drm and conditionally transformed with v-mos-expressing Moloney murine sarcoma virus (Mo-MuSV) rapidly undergo apoptosis when shifted to the non-permissive temperature. These results indicate that drm represents a newly identified gene which appears to play a role in cell growth and tissue-specific differentiation.

Identification of an mRNA expressed in revertant cells but repressed in v-mos-transformed rat fibroblasts. To identify genes expressed in F-1 revertant cells, but not in v-mos-transformed parental cells (DTM), differential display analysis (25) was performed, using oligo dT-selected RNA isolated from rapidly-growing DTM and F-1 cells. Eight cDNAs showing differential intensities between DTM and F-1 mRNAs were identified and used to probe Northern blots containing poly(A)+ RNA from DTM and F-1 cells. Only one exhibited differential mRNA expression, detecting a 4.4 kb RNA expressed in F-1 cells, but absent in DTM cells. Analysis of this cDNA, designated drm (for down-regulated in v-mos transformed cells), revealed a 691 bp sequence, which included a consensus polyadenylation signal (AATAAA) located 20 bp upstream from the poly(A) tail, as well as the 5′ and 3′ primers used for PCR. A search of nucleotide sequences compiled in the GenBank data base showed no significant similarities to known genes.

Repression of drm mRNA Expression following Cell Transformation.

To establish a correlation between repression of drm gene expression and the transformed cell phenotype, the hybridization of drm cDNA to RNA from normal and transformed REF-1 cells was analyzed. Drm was expressed at similar levels in both REF-1 and revertant F-1 cells, but its expression was completely repressed in REF-1 cells transformed by the v-ras, v-raf, v-src and v-fos oncogenes. These results demonstrated that repression of drm expression was not restricted to transformation induced by v-mos.

Because the initial identification of drm was based on its expression in the F-1 revertant and it had been previously shown that F-1 cells could be transformed by v-ras and v-src, the effect of expression of these oncogenes in F-1 cells on drm expression was analyzed. F-1 cells expressing and transformed by v-ras and v-src did not contain drm transcripts detectable by Northern blot analysis, while in contrast, F-1 cells infected with the v-mos expressing MSV-124 show levels of drm RNA essentially identical to uninfected F-1 cells or REF-1 parental cells. Since it had been previously shown that superinfection of F-i cells with additional copies of v-mos did not induce transformation (41), these results are consistent with the hypothesis that drm expression is down-regulated following oncogene-mediated transformation.

To further analyze the correlation between drm expression and the transformed phenotype, REF-1 cells transformed by a temperature-sensitive (ts) isolate of Moloney murine sarcoma virus (Mo-MuSV tsl 10) (3) were used. These cells (ST33c) are transformed at 34° C., but express a phenotypically normal, non-transformed phenotype at 39° C. (42). Analysis of RNA extracted from cells maintained at both temperatures indicated that drm RNA was synthesized at 39° C. in the absence of the v-mos protein and was markedly decreased at 34° C. Taken together, these results further indicate that in REF-1 cells repression of the drm RNA expression correlates with the transformed phenotype. The results with ts MuSV-transformed cells and the F-1 revertant indicate that drm expression is directly or indirectly modulated by the v-mos oncoprotein and its transforming functions.

Drm is a novel gene. To fully characterize the drm gene and its product, rat fibroblast and rat kidney cDNA libraries were screened and five independent overlapping cDNA clones were isolated, which covered ˜3820 bp of drm mRNA. Southern blot analysis indicated that the drm sequence is derived from a single gene spanning at least 12 kb and is not rearranged in either DTM, which does not express drm, or in the F1 revertant.

The 3820 nucleotides of cloned cDNA is shorter than the apparent size of the RNA identified in REF-1 cells, suggesting that the isolated clones may not include the entire drm mRNA sequence. However, this cDNA does contain a single long open reading frame (ORF) beginning at nucleotide 130 and terminating with an in-frame stop codon at nucleotide 693. Translation is predicted to start at the first in-frame methionine at nucleotide 139 within a favorable translation initiation context (A at −3, C at −4, G at −6 and A at +4) (22,23). Thus, the characterized drm cDNA consists of 138 bp of 5′ untranslated (UTR) sequence (65% GC), a 552 bp coding region and 3130 bp of 3′ UTR containing a consensus polyadenylation signal AATAAA located 21 nucleotides upstream from the poly(A) tail.

The major ORF contained in the drm cDNA would be predicted to encode a 184 amino-acid polypeptide with a calculated molecular weight of 20,682. The presumptive drm gene product is highly basic (7.61% arginine, 8.7% lysine and 2.17% histidine), with the NH₂-terminal half containing a leucine-rich hydrophobic domain located between amino acids 4 and 24, whereas the carboxy-terminal moiety is characterized by the presence of nine cysteines. The presence of an amino-terminal hydrophobic domain suggested a possible membrane localization of the protein and analysis of the drm deduced amino-acid sequence using the TMbase database of transmembrane proteins (Lausanne) indicated a high probability that this protein could form a transmembrane helix in this region. Examination of the predicted sequence also identified two potential nuclear localization signals which fulfill the motif K(R/K)×(R/K): KPKK (amino acids 145-148) and KKKR (amino acids 166-169), two protein kinase C phosphorylation sites (TER, amino acids 84-86 and TKK, amino acids 165-167) and three cAMP and cGMP-dependent protein kinase phosphorylation sites (KKGS, amino acids 26-29, KKFT, amino acids 147-150 and KRVT, amino acids 168-171).

Comparison of the drm amino-acid sequence to the GenBank and EMBL data bases using FASTA program, showed that the drm protein exhibits an overall similarity of 30% with the rat and human DAN gene product, which expresses tumor-suppressive properties (28,29). Using the BLAST program, a 52% similarity was detected between the carboxy-terminal cysteine-rich half of drm, the central region of the DAN protein and the carboxy-terminal region of the Xenopus protein Cerberus (CER), a head-inducing secreted factor expressed in the anterior endoderm of Spemann's organizer (4). Further analysis also revealed similarity to the carboxy-terminal cysteine-rich end of the human MUC2 intestinal mucin (16). The nine cysteines of the drm are also present in DAN, CER, and MUC2 gene products at similar amino-acid intervals. This alignment generated the cysteine motif CX13CX(8-9)CX3CX(14-18)CX2CX13CX(15-18)CXC. Within this motif several amino acids are conserved, suggesting that proteins containing this domain could be members of a related family.

Characterization of the drm Gene Product. In vitro transcription/translation of the ORF-containing 2.1 kb EcoRI fragment and 730 bp BamHI/KpnI fragment of drm cDNA confirmed that the presumptive open reading frame could express a protein of approximately the expected size. To further characterize the drm product, an anti-peptide polyclonal rabbit antibody directed against amino acids 79 to 92 of the rat drm protein was generated. In order to assess the specificity of the antisera, an expression vector was constructed, synthesizing an epitope-tagged drm protein by introducing a DNA fragment encoding the nine-residue epitope of influenza virus hemagglutinin HA1 at the 3′ end of the coding region. The pSVL expression vector containing this fusion was used to transfect Cos-7 cells and cell lysates were prepared 48 hrs later, immunoblotted on nitrocellulose filter and incubated with the drm antisera. A band with a predicted molecular weight of 21.4 kDa was detected and the same band was revealed with the monoclonal antibody against HA tag. It was not detected when lysates were exposed to 990 antisera preincubated with peptide against which this antiserum was raised nor in lysates of cells transfected with an empty vector. A protein of the same molecular weight was detected in HA-drm-transfected Cos-7 lysates immunoprecipitated with 990 antiserum and blotted with anti-HA sera and this precipitation could be blocked by the homologous 990 peptide.

To identify the endogenous drm protein, total lysates from various cells were analyzed by Western blotting. Low levels of a 20.7 kDa protein were detected in primary embryonic rat fibroblasts and in REF-1 cells. Analysis of drm protein expression in ST33 cells, conditionally transformed by v-mos, showed good correlation with drm-specific RNA expression. The protein was not detected in lysates of transformed cells at 34° C., but could be seen in cell lysates prepared 48 hrs after shifting the cultures to the non-permissive temperature. Drm protein was not detected in lysates of v-mos-transformed DTM cells.

Drm RNA is Expressed in a Tissue-Specific Fashion in Adult Rats.

To further characterize the drm gene and its possible function, the expression pattern of drm was examined in rodent tissues. Northern blot analysis of polyA+ RNA extracted from adult rat tissues (Sprague-Dawley) showed that the drm gene was expressed in brain, kidney, spleen, testis and lung and was not detected in heart and skeletal muscle. Highest levels were seen in kidney, testis, brain and spleen, while levels in the liver and lung were significantly lower.

To investigate whether drm expression was specific for any particular cell type, tissues from the same strain of rat were analyzed by in situ hybridization using sense and antisense drm riboprobes. In situ expression patterns in general correlated well with the Northern analysis, but drm RNA appeared to be predominantly expressed in differentiated cells (e.g., neurons in brain, type 1 cells in lung, goblet cells in intestine). In all cases the control sense probe showed no detectable hybridization.

The brain exhibited ubiquitous expression of drm RNA. High levels of drm expression were found in both neurons and glial cells of the brain cortex, while in the cerebellum, drm RNA was strongly expressed in all cells of molecular and granular layers. Its expression was significantly weaker in Purkinje cells.

In the kidney, drm RNA was found in epithelial cells of the proximal and distal tubules in the cortex, medullae and papillae. Very strong signals appeared to be localized in the nuclei of the epithelial cells.

In the small and large intestine, the drm gene was predominantly detected in goblet cells and specifically in the most differentiated goblet cells (on the tip of the villi in small intestine and the base and neck of the crypt in large intestine). However, some goblet cells in the crypt of the small intestine were also found positive for drm expression.

In the lung, the drm expression was localized to the nucleus of type 1 epithelial cells lining the alveoli. Type 1 cells are known to be terminally differentiated from their precursor type 2 cells (6). Drm was not expressed in every type 1 cell, which could indicate a possible correlation of drm expression with the stage of cell differentiation. A few endothelial cells of the airways and a number of macrophages also expressed drm RNA, while in the spleen, drm RNA was detected only in megakaryocytes and in agreement with the results of Northern blot analysis, drm hybridization was not detected in liver, heart and skeletal muscle.

drm Blocks Colony Formation by Normal, but Not Transformed Cells.

To determine the biological effect of drm overexpression in vivo, a portion of the drm cDNA containing the full-length ORF was inserted into the neo-containing pMEX expression vector (21). This construct, as well as the empty vector, was introduced into REF-1 and DTM cells and G418-resistant colonies were counted after 2-3 weeks. Colony formation was inhibited 30-fold when REF-1 cells were transfected with the drm expression vector. The mos-transformed DTM cell colony formation was not affected. Similar results were also seen in CHO cells, indicating that inhibition of colony formation is not specific to REF-1 cells. Analysis of independent, drm-transfected G418-resistant clones of REF-i cells showed that all surviving clones expressed very low or undetectable levels of exogenous drm mRNA, suggesting that survival may select for cells expressing low levels of drm. In contrast, DTM cells, which showed no inhibition of colony formation, exhibited high levels of exogenous drm expression. In some cases, expression of endogenous drm RNA was also increased in DTM cells expressing exogenous drm, suggesting a possible autoregulation loop of drm expression.

Since oncogene-transformed stable cell lines had shown down-regulation of drm expression (see above), the interactions between transforming oncogenes and drm were further investigated by co-transfecting REF-1 and CHO cells with drm and the activated (38) ras oncogene. Consistent with previous results with DTM cells, co-transfection of drm with the ras oncogene did not suppress morphological transformation. However, co-transfection of ras with drm reversed the drm-dependent inhibition of colony formation both in REF-1 cells (84% of the control) and in CHO cells. The level of exogenous drm RNA in 5 of 6 G418-resistant clones co-transfected with pMEXdrm and ras was increased. These data are consistent with the hypothesis that high levels of drm inhibit the growth or viability of normal cells, but that transformed cells are resistant to this inhibitory effect.

Conditionally-Transformed Cells Expressing Exogenous drm Undergo Apoptosis at the Non-Permissive Temperature.

Since transfection of non-transformed rat and hamster cells with drm expression vectors leads to the inhibition of cell growth, stable cell lines expressing high levels of drm could not be obtained for molecular and biological analysis. In order to overcome this problem, conditionally-transformed ST33c cells were used to investigate the effects of drm overexpression. When v-mos is functional (34° C.) and ST33c cells are transformed, transfection of pMEXdrm vector does not affect the efficiency of colony formation in comparison to control vector. These results are consistent with the data for DTM cells and for REF-1 cells co-transfected with pMEXdrm and ras, showing that the presence of transforming oncogene blocks the inhibitory effect of drm. In contrast, at 39° C., the percentage of survived colonies following pMEXdrm transfection was significantly lower than that observed in control vector-transfected ST33c cells.

To analyze how drm overexpression blocks cell growth and colony formation, G418-resistant colonies of transfected ST33c cells were isolated at 34° C. and tested for the expression of drm. Pools of G418-resistant cells expressed elevated levels of drm RNA similar to those seen in transfected DTM or ras-transformed cells. These transfected pools grew like the parental ST33c cells at 34° C., when v-mos is expressed, but rapidly lost viability after shifting to 39° C., and colony-forming ability was significantly reduced. This is consistent with the fact that, as previously shown, v-mos is not expressed in these cells at 39° C., and thus cannot neutralize the effects of the high level of exogenous drm in these cells. The morphological changes seen in these cells at 39° C. resemble those of cells undergoing apoptosis, including cell shrinkage, cell membrane blebbing and loss of cell-cell contact and adhesion to the substrate. Furthermore, drm-expressing ST33c cells exhibited nuclear fragmentation and condensation within 24 hrs of a shift to 39° C., while no such fragmented nuclei were observed in these cells cultured at 34° C. or in REF-1 cells at either 34° or 39° C. It was observed that 15-30% of the ST33c cells expressing drm at 39° C. exhibited fragmented, condensed nuclei, while only 5-6% of the control ST33c cells manifested similar changes following a shift to 39° C. DTM cells, transfected with drm and containing two copies of v-mos (ts- and w.t. v-mos) also showed 5-7% fragmented nuclei at 39° C., which could represent the background level for ts v-mos-transformed cells shifted to 39° C. Apoptosis of drm-expressing ST33c cells at 39° C. was also confirmed by agarose gel electrophoresis of genomic DNA, which showed significant fragmentation only in the cells shifted to 39° C. Furthermore, the relative fraction of cells undergoing apoptosis were seen to correlate with the level of drm expression in a series of individual clones of ST33c cells transfected with drm. Taken together, these data suggest that cells expressing high levels of drm undergo apoptotic death in the absence of oncogene-induced transformation.

### Example II.

### Isolation and Characterization of Human drm Gene and Gene Product

Cell Culture, Transfection and Synchronization.

All human cells, including normal diploid fibroblasts, were grown in HG-DMEM. CHO cells were grown in F12 medium. All media was supplemented with 10% fetal calf serum (FCS) (Atlanta Biological, Norcross, Ga.) and cells were maintained at 37° C. with 10% or 5% CO₂ (for CHO cells). For serum starvation, medium was changed to 0.1% FCS when cells were subconfluent and cells were left in this medium for 72 hours. For density-dependent inhibition, cells were plated at 10⁴/cm² in 10% FCS. Twenty-four hours after plating, the medium was changed every two days. Exponentially-growing cells are cells cultured for 24 hours in 10% FCS. Human cells were synchronized as described previously (71). Briefly, IMR90 or Hem cells were grown in MEM α modification (Gibco, BRL) with 0.1% FCS for 72 hours prior to replacement with 10% FCS. Nine hours later, hydroxyurea (HU) (Sigma) was added to a final concentration of 0.5 mmol/U to arrest the cells at the G₁/S boundary. After nine hours of HU blockade, the complete medium was added and cells were taken for protein and flow cytometry analysis (FACS).

Transient transfections of cells were performed by using Lipofect AMINE or Lipofect AMINE PLUS (for IMR90) (Life Technologies) as specified by the manufacturer.

FACS.

For cell cycle analysis of human cells, at hourly intervals, the cells were harvested and washed with PBS, the number of cells was counted and 1×10⁶ cells were processed for flow cytometry. Cells were suspended in PBS with 0.05% Triton X100. DNase-free RNase (200 U/ml, Boelringer Mannheim) was added for 30 minutes at 37° C. and then the cells were washed twice. Propidium Iodide (PI) was added to a final concentration of 50 mg/ml (71). The cells were examined for DNA content with FACScan flow cytometer (Coulter Epic S′ Profile II, Coulter Corp., Miami, Fla.) and the percentages of cells in G₀/G₁, S and G₂/M phases were determined with MultiPlus AV version 3.0 software.

To analyze the cell cycle of sorted cells, CHO cells were transfected with pEGFP or pDRM-GFP. At 24 hours after transfection, cells (50×10⁶) were harvested by trypsinization and EGFP-expressing cells were recovered by fluorescence-activated cell sorting (FACS). Cells were fixed in 70% ethanol at 4° C. and recovered by centrifugation. The fixed cell pellet was resuspended in 0.9 ml of PBS with 0.1% BSA and RNaseIIIA (200 U/ml) was added for 15 minutes at RT. DNA was stained with PI and examined with FACScan flow cytometer (Coulter Epics 753, Coulter Corp., Miami, Fla.), and the percentages of cells in G₀/G₁ S and G₂/M phases were determined with MultiPlus AV, version 3.0 and Elite software programs.

Northern Blot Analysis.

For Northern blot analysis, Human Multiple Tissue Northern (MTN) blots (I-II), (II-III) (Clontech) and human RNA master blots (Clontech) were used. The blots were probed with a radiolabeled human DRM-specific probe. Hybridization and washing conditions were in accordance with the manufacturer's instructions.

Total RNA was extracted from cultured cells by RNAzol B (Tel-Test, Inc., Friendswood, Tex.), and hybridized with a human DRM probe as described previously (Topol et al., 1992).

Screening of a cDNA library. To determine the DRM cDNA sequence, a human small intestine 5′-stretch cDNA library in λgt11 (Clontech) was screened using 5′ sequences of rat drm (Cl 7ZAP) (65). Five clones were isolated. The largest one (3.2 kb) was amplified and analyzed. Both strands of the double-stranded plasmid DNA were sequenced by primer walking using the dideoxy chain dye terminator method with Amplitaq DNA polymerase, FS (Perkin Elmer). The sequencing products were analyzed on an ABI prism 377 DNA sequencer (Perkin Elmer). The nucleic acid sequence of the DRM gene was analyzed using the GCG package (University of Wisconsin).

Rapid Amplification of cDNA Ends (RACE).

For 5′-RACE, 1 μg of total RNA from human diploid fibroblasts was mixed with the DRM-specific primer and reverse transcribed with 200 U of Superscript II reverse transcriptase (Gibco/BRL) at 42° C. for 30 minutes according to the manufacturer's protocol. The final products were subcloned into the EcoRI site of the pCRII plasmid and sequenced with vector-specific oligonucleotide primers.

Construction of EGFP-DRM Fusion Expression Vector.

The coding region of the DRM gene was PCR amplified from a cDNA using Ultima DNA polymerase (Cetus) and primers containing a BamHI restriction site. The primers used were 5′ (CGGGATCCAGAATGAATCGCACGGCATAC) (SEQ ID NO:11) and 3′ (GCGGATCCTTAATCCAAGTCGATGGATATGC) (SEQ ID NO:12) (primers from Biosynthesis, Inc., Lewisville, Tex.). The PCR product was digested with BamHI and inserted into an EGFP-C1 expression vector (Clontech) which was digested with BamHI and treated with Shrimp Alkaline Phosphatase (Boehringer Mannheim).

Western Blot Analysis.

Cells were lysed in boiling 2× SDS sample buffer. Equal amounts of lysates (determined by Bradford protein staining reagent, Bio-Rad) were electrophoresed on 4-20% SDS-PAGE and transferred to Hybond ECL nitrocellulose membrane (Amersham). Equal loading and transfer was confirmed by staining reversibly in 0.2% Ponceau-6% TCA (Sigma). The membranes were incubated first with the appropriate primary antibody and then with horseradish peroxidase-labeled secondary antibodies (Amersham). Antibodies were detected by using the ECL detection system (Amersham) or the Super Signal CL-HRP Substrate System (Pierce) and visualized by using Kodak XAR-5 X-ray film. Western blots were stripped for reprobing with other primary antibodies as specified by the manufacturer (Amersham).

Probes and Antibodies.

cDNA probes were obtained from the following sources: rat NSE cDNA (79) from Dr. Gregor Sutcliffe; human GFAP cDNA was purchased from the ATCC. Polyclonal antibodies (e.g., 990), which recognized DRM, were described previously (65). Other antibodies used in this study were specific for p27^{Kip1}, p21^{Waf1}, cyclin E (Transduction Lab., Lexington, Ky.), cyclin E (Ab-1, Oncogene Research), cyclin E (M-20, Santa Cruz Biotechnology; SC35), cyclin D1 (R-124, Santa Cruz), GFP (Clontech), p53 (PAb122, D01, Pharmingen), pCdK2 (M2, Santa Cruz), PhosphoPlus Rb/Ser 795), antibody kit (New England Biolabs), β-actin (Chemicon).

BrdU Incorporation.

The effect of DRM expression on bromodeoxyuridine (BrdU) incorporation was determined in CHO cells growing asynchronously in F-12-10% FCS. Cells were plated at 10,000 cells/ml on coverslips and after 24 hours were transfected with 5 μg of either pEGFP, or pDRM-EGFP. Twenty-four hours after transfection, the medium was changed and cells were incubated with BrdU labeling reagent for a further 12 hours according to the supplier's (Amersham) instructions. After labeling, coverslips were washed in PBS and cells were fixed in 3% paraformaldehyde. Incorporated BrdU was detected with a monoclonal anti-BrdU antibody (Boehringer Mannheim) by immunocytochemistry.

Immunocytochemistry and Immunofluorescence.

Fixed cells on coverslips were washed twice with PBS and treated with 0.1M glycine in PBS for 5 minutes at RT, followed by treatment with 0.1% Triton X-100 in PBS for 4 minutes at RT and 50 mM NaOH for 10 seconds. Co-localization of DRM with the speckles was analyzed by immunofluorescence with a monoclonal antibody SC35 (80) and a rhodamine-conjugated, goat anti-mouse immunoglobulin G secondary antibody (Kirkegaard and Perry Labs., Gaithersburg, Md.). Coverslips were mounted and examined with a fluorescence microscope.

Chromosomal Mapping of DRM Gene.

A somatic cell hybrid panel (Oncor) was hybridized with a ³²P-labeled 1.2 kb human 5′ DRM cDNA fragment according to the manufacturer's protocol.

In order to localize the DRM gene on human chromosomes, a special probe was prepared by PCR using primer #197 (position 2934-2955): 5′TCATTACATCATCAGTGACTCG3′ (SEQ ID NO: 20) and #195 (position 3131-3152): 5′CAGATTTGGCTCAAGTAAAGAG3′ (SEQ ID NO:21). The result of this reaction was a fragment (195 PCR) representing 218 bp specific for the human DRM sequence. Chromosomal localization of the 195 PCR product was accomplished using two panels of somatic cell hybrids. The first was a hybrid mapping panel #2 from the Coriell Institute for Medical Research. This is a collection of 24 human X hamster cell lines. All but two of these hybrids retain a single, intact human chromosome. The second panel is the GenBridge 4 radiation hybrid panel available from Research Genetics (73). PCR reactions were carried out as follows. Twenty-five ngm of hybrid or control DNA were amplified in a 10 μl volume in a reaction buffer consisting of 10 mM Tris-HCl, pH 8.3, 50 mM KCl, 1.5 MM MgCl₂, 200 μM of each dNTP, 1 pmol of each primer and 0.001 units of Taq Gold (Perkin Elmer) polymerase. The PCR cycling conditions were as follows: an initial 94° C. denaturation step for 10 min followed by 35 cycles of 94° C. denaturation for 30 sec, 60° C. annealing for 1 min and a 72° C. extension step for 1 min, followed by a 72° C. heating for 5 min. PCR products were run out in 1.2% agarose gels and stained with ethidiurn bromide. After scoring each radiation hybrid for the presence or absence of the PCR product, the resulting vector was sent by electronic mail to the MIT/Whitehead Institute Genome Center for analysis.

Subcellular Fractionation.

Subcellular fractionations were prepared as described previously (89). The fractionation protocol was first verified on COS7 cells transfected with expressing vector pGFP (Green Fluorescent Protein) to confirm the correct distribution of control proteins. Cells grown on 100 mm culture dishes as a monolayer were washed and scraped in PBS, centrifuged and resuspended in hypotonic buffer A (10 mM Hepes, 1.5 mM MgCl₂, 10 mM KCl, 0.5 mM PMSF) (18). After 15 min of swelling on ice, cells were homogenized carefully by 20-25 strokes in a Dounce homogenizer (Type B pestil) to break the cells. This procedure was carefully monitored by fluorescence microscopy for staining of “broken cells” with propidium iodate (PI) to ensure >90% lysis of the cells without breakage of the nuclei. After centrifugation at 800 g for 10 min (4 C), the pellet, consisting of a mixture of unbroken cells and crude nuclei, was designated the low speed pellet and was processed further. The supernatant was collected and subjected to further centrifugation at 100,000 g for 30 min. The resulting supernatant contained soluble protein and was designated the cytoplasm fraction (C). The pellet was considered the particular fraction (P). The low speed pellet was washed in a large volume of buffer A and resuspended in 2 vol buffer A′ (buffer A supplemented with 0.5 mM DTT and 1% NP-40) of the initial cell pellet. After incubation on ice for 10 min, the sample was centrifuged, the supernatant was removed and cleared as described above, generating a pellet (N) and supernatant fraction. This resulting supernatant, containing soluble cytoskeleton proteins, was designated the skeleton fraction (Sk). The pellet (Pk) represented unsoluble cytoskeleton fraction. The remaining nuclei were again washed in Buffer A′, pelleted at 10,000 g, resuspended in 4 vol 2×SDS-loading buffer, sonicated three times for 20 s, and boiled for 10 min. Each subcellular fraction was then assayed for its protein content and an equal amount of total protein (40 g) was loaded on the gel.

Molecular cloning of human DRM. A new gene sequence (drm) (GenBank Accession No. Y10019) has been previously identified, based on differential display analysis of v-mos-transformed rat fibroblasts and their flat revertant (65). Zoo-blot analysis indicated that the drm sequence is present not only in rodents (rat and mouse) but also in humans. To isolate the human drm homolog a human small intestine 5′-stretch cDNA library was screened with a probe that encompasses the coding region of rat drm to obtain a full-length of cDNA insert. Among the positives, the longest clone (3.2 kb) found included the majority of the open reading frame (ORF) of drm. To extend the 5′ end of the obtained clone the 5′ RACE-PCR technique was applied on RNA extracted from primary human diploid fibroblasts and extended the clone for an additional 200 bp. This 3.411-nucleotide sequence, excluding the poly(A) tail, contains one large ORF from position 130 to 683, which encodes a protein of 184 amino acids (Mᵣ, 20, 682). A single ORF was found, with the ATG translation initiation site located at position +1 and the TAA stop codon at position +553. This ORF is preceded by a stop codon (TAG) at position −105. This was designated as the translation start site as there was no ORF upstream of this codon and it includes a Kozak consensus sequence for translation initiation (74).

Comparison of the human and rat DRM cDNAs revealed that these two cDNAs have a highly-related sequence in the coding region (˜86% identity), but they are divergent in 5′ and 3′ untranslated sequences (UTR). In the 5′ UTR, the hu-DRM contains two long stretches of GC (19 and 11 nucleotides) at −100 and −80, respectively. Comparison of the rat and human DRM amino acid sequences demonstrated a high conservation (181/184 amino acids) between rodent and man. Like rat drm, human DRM has two putative nuclear localization signals near the C-terminus (amino acids 145-148 and 166-169), a cysteine-rich region (93-178) and several sites for phosphorylation by protein kinase C (amino acids 84-86, 165-167), cyclic AMP and cyclic GMP-dependent protein kinases (amino acids 26-29, 147-150 and 168-171), respectively. This striking identity implies that the overall three-dimensional shapes of the two proteins are very similar. This may in turn indicate that the two proteins are functionally equivalent.

DRM maps to human chromosome 15. Southern blot analysis of BamHI-digested DNA from mouse-human somatic cell hybrids harboring a single human chromosome was carried out using 1.2 kb human DRM 5′ cDNA as a probe. One single band was detected in the DNA from hybrid cells harboring human chromosome 15. The DRM gene was also localized by PCR analysis.

Successful amplification of the 218 bp human 195 PCR product was obtained in control human, but not in hamster DNA. Amplification of the Cornell hybrid DNA indicated that this gene was located on chromosome 15. Analysis of the radiation hybrid data placed this PCR product 23.32 cR distal to the chromosome 15 reference marker WI-5590 and one cR distal to marker D15S144. This is a position about 59cR from the top of the chromosome 15 radiation hybrid map, about 23 cM from the top of the linkage map and corresponds to a cytogenetic location of 15q11-q13 (73,75).

DRM is a Secreted Protein that Remains Cell Associated.

The cellular localization of DRM has also been analyzed using both cell fractionation and immunofluorescence microscopy. COS cells transfected with pHA-DRM were separated into multiple subcellular fractions and the relative distribution in the particulate (P), soluble cytoplasmic (C), nucleus/cytoskeleton-associated soluble (Sk) and insoluble (Pk), and pure nuclear (N) fractions, was determined by western blot analysis with anti-DRM antibodies. The protein was detected predominantly in the insoluble particulate fraction (P) and the detergent-extracted soluble and insoluble cytoskeleton-associated fractions (Sk and Pk). Quantitation of these results by densitometry indicated that over 70% of DRM was localized in the insoluble membrane and cytoskeletal fractions (Pk and Sk), while 17% was found in the cytoplasmic (C) fraction and 9% in the nucleus (N). To verify the subcellular fractionation, the same filters were blotted with antibodies recognizing the membrane localized p145 c-met protein. As expected, c-met was found predominantly in the insoluble membrane fraction (fraction P).

To confirm and further analyze the distribution of DRM, DRM localization in COS cells overexpressing pHA-DRM was investigated by immunofluorescence. Transfected cells were fixed with paraformaldehyde and probed with DRM polyclonal antibodies and Oregon green 488 conjugated anti-rabbit secondary antibody. Alternatively, the cells were permeabilized following fixation and subsequently treated with antibodies. Permeabilized cells exhibited a diffuse, fiber-like network of staining, suggestive of a localization in the endoplasmic reticulum/Golgi complex, and some cells also exhibited a distinct perinuclear staining, which could be the site of DRM synthesis. To confirm this intracellular localization, monoclonal antibodies directed against the Golgi-specific p58K protein, specifically localized on the cis/medial side of the Golgi apparatus were used. The results showed that both DRM and p58K co-localized in the Golgi stacks.

In contrast, non-permeabilized cells showed a clumped, punctate pattern that appeared to surround the outer surface of the cell membrane, indicating the presence of DRM on the external cell surface. Analysis of live, unfixed cells showed a similar pattern. A similar subcellular distribution of DRM was observed in COS cells by using anti-HA antibodies and in rat cells expressing the endogenous protein, although in the latter, intracellular staining was predominantly cytoplasmic and perinuclear.

Taken together, these results indicate that DRM is transported through the cell membrane to the outer surface of the cell. To confirm that the hydrophobic region was responsible for DRM's entrance into the secretory pathway, COS7 cells were transfected with pHA-DRM-21N and the localization of the truncated protein was determned by using anti-DRM and anti-HA antibodies. The truncated protein was found to be exclusively intranuclear, consistent with the fact that the protein also contains 2 NLS's (amino acids 147-150 and 168-171), and indicating that the two NLS signals are functional. As expected, surface staining was not observed when these live or nonpermeabilized cells were treated with antibodies, indicating that DRM is unable to be secreted in the absence of the 21aa amino terminal region.

Results of both cell fractionation and immunofluorescence indicated that DRM is a secreted protein. However, the protein was not detected in culture fluids of either COS7 cells overexpressing DRM, CHO cells expressing transfected DRM, or rat fibroblasts expressing the endogenous protein. The failure to detect soluble DRM was not technical because the reconstitution experiments demonstrated that the protein was detectable under these conditions. To test the possibility that the secreted DRM protein remains associated with the external cell surface, pHA-DRM transfected COS cells were treated with acidic buffer, conditions which have been shown to dissociate non-covalently bound polypeptide ligands from their receptors. This treatment significantly reduced the amount of detectable glycosylated DRM, whereas it did not apparently decrease the amount of the faster migrating non-glycosylated form.

When transfected CHO cells were treated with acid buffer, the amount of DRM proteins significantly decreased and the upper glycosylated band was no longer detectable Treatment of both transfected cell lines with trypsin decreased the amount of glycosylated DRM. Incubation of the same membranes with anti-EGF-R or actin antibodies showed that the levels of these two proteins were not affected by these treatments. To confirm that intact DRM protein had been removed from the outer plasma membrane, proteins were concentrated in the acid wash by acetone precipitation and analyzed by immunoblotting. The protein was detectable in the acetone-precipitated sample at low levels, migrating as multiple bands.

The DRM/GFP Fusion Protein is a Nuclear Protein.

In order to localize the DRM product a vector containing the fusion EGFP-DRM insert under a CMV promoter was constructed. CHO cells were transfected with the expression vectors encoding only green fluorescent protein (PEGFP) or fusion EGFP-DRM (pEGFP-DRM). Comparison of the fluorescence from the EGFP alone with that of the EGFP-DRM fusion showed that the chimeric protein was exclusively localized in the nuclei of CHO cells. EGFP-DRM product was also found to be localized in the nuclei of HeLa, SaoS, Cos-7, and normal human fibroblasts transiently transfected with EGFP-DRM vector. The pattern of distribution of EGFP-DRM in the nuclei varies, including, predominantly, structures of punctate shape (dots), but very rarely, in single cells, uniformly diffused nuclear distribution could be seen. Amounts of nuclear dots could be different: from a few large to numerous small ones. Taking into account this specific pattern of distribution in the nuclei which resemble a speckled pattern, experiments were conducted to co-localize DRM with other known subnuclear structures such as non-snRNA splicing factors (SC35) (81). In immunofluorescence labeling experiments with monoclonal anti-SC35 antibody for transiently-transfected Cos cells with GFP-tagged DRM, SC35 and DRM did not co-localize, but in several nuclei these two proteins did occupy the same regions. DRM did not co-localize with nucleoli, as determined by co-transfection of HeLa and CHO cells with blue fluorescent protein (BFP)-tagged Rev, which is known to have nucleoli localization (82).

Distribution of DRM Transcript in Normal Human Tissues.

To characterize the level of endogenous DRM mRNA expression in human tissues a multitissue poly(A)+ RNA Northern blot (Clontech) was hybridized with a 1.2 kb 5′ end hu-DRM cDNA fragment. On a Northern blot, a single transcript of approximately 4.4 kb was detected in several tissues, including the prostate, ovary, small intestine, colon, brain, skeletal muscle and pancreas. The highest level was seen in the small intestine and colon; however, in the brain and ovary, DRM expression was also high based on normalization of poly(A)+ RNA for β-actin. No specific mRNA was detected in spleen, thymus, heart, lung, liver, placenta and peripheral blood leukocytes. This expression pattern of DRM is different from the expression pattern of the rat DRM, but in both, the brain was positive for DRM expression. To expand the information about the tissues where DRM is expressed, the human RNA Master Blot was used, whose data confirmed the previous one, but showed that DRM also is expressed in colon, stomach, appendix and lymph nodes.

To investigate whether DRM expression could be detected during human embryonal development, a human fetal multiple tissue Northern blot (Clontech) was analyzed, demonstrating that DRM is highly expressed only in fetal brain. Previously, using in situ hybridization, it was shown that the rat adult brain exhibited ubiquitous expression of drm RNA (65). The expression of human DRM in different regions of the human brain was examined. The analysis of several human brain regions revealed widespread expression of DRM, although with different intensity. Based on normalization for β-actin, the highest abundance was found in the putamen, corpus callosum, substantia nigra, caudate nucleus and cerebral cortex. A high level of expression was found in the medulla, thalamus and subthalamic nucleus, and a low level of expression was detected in the amygdala, spinal cord and frontal lobe,

Based on previous data in rat (65) where a high level of DRM expression was detected in neurons, a specific marker for neurons, neuron-specific Enolase, NSE (79) and glial fibrillary acidic protein, GFAP (84), was used as a marker for astrocytes, to evaluate the connection of DRM expression with these two markers. In corpus collosum, the major expression of DRM-specific RNA coincides with a high level of GFAP expression, which is specific for astrocytes. At the same time, in the cerebellum and cerebral cortex, a high level of DRM expression coincides with expression for a neuron marker, which supports the data obtained with in situ hybridization earlier. In putamen, temporal lobe, frontal lobe and occipital pole, all DRM expression coincides with NSE, which suggests that DRM is expressed in differentiated neurons in the adult human brain.

DRM Expression in Normal and Transformed Cultured Cell Lines.

Since DRM was initially isolated as a gene whose expression was down-regulated in v-mos-transformed cells, more than 70 human tumor and normal diploid cell lines were screened for DRM expression. The DRM transcript was found predominantly in normal human diploid fibroblasts of different origins (10/10) and in normal human astrocytes, but was not detected in normal melanocytes, normal mammary glands and the HUVEC cell line. DRM was not detected in essentially all tumor cell lines examined. These results raised the possibility that the tumorigenic phenotype is incompatible with the continued expression of DRM and that down-regulation of DRM is necessary as a step in transformation. To investigate this assumption, the level of DRM expression in cells was examined at different stages of transformation. We established a system containing primary, immortalized and transformed rat fibroblasts, isolated RNAs and proteins from the cells and determined the level of DRM expression. Primary rat fibroblasts were shown to contain a high level of DRM on RNA and protein levels; in immortalized cells (REF-1) the level of DRM was decreased 2-fold. Finally, in transformed rat fibroblasts the DRM expression was not detected at either RNA and protein levels. These results demonstrate that the level of DRM expression is tightly regulated and may reflect both the state of transformation and/or proliferative activity.

To assess the expression of DRM during density-dependent growth inhibition, normal human fibroblasts were seeded in 10% FCS and the medium was replaced every second day with fresh 10% FCS. Northern blot analysis showed DRM induction after 6 days of density inhibition of growth when cells entered quiescence. Most striking is the fact that the expression of DRM-specific RNA was amplified up to 10-fold in density-arrested human fibroblasts. These data demonstrate that human fibroblasts accumulate DRM mRNA when they exit the cell cycle and enter a quiescent state as they grow to high density.

Modulation of DRM expression during the cell cycle. Since DRM expression was found to increase in primary rat fibroblasts when proliferation is under strong regulation and in human fibroblasts under density-mediated arrest in G₀, the DRM protein level was examined for changes during the cell cycle. Normal human diploid fibroblasts (IMR90 and HEM cells) were synchronized by serum starvation for 72 hours in minimum essential medium alpha modification (71) followed by arrest at the G₁/S boundary by hydroxyurea (HU) blockade and subsequent release of this block with fresh complete medium. Lysates were prepared at different times after HU blockade release and samples were analyzed by Western blotting with anti-DRM antibodies. It appears that the level of DRM proteins change in a cell cycle-dependent manner. The highest amount of DRM was observed during G₀ when the cells were arrested by serum deprivation for 72 hours. The level of DRM protein was found to decrease 3-fold as cells reached the G₁/S boundary, to be low during the S phase and to increase again in the end of the S phase and as cells entered the G₂/M phase. Cyclin E expression was used as a control for cell cycle progression (78). The changes in DRM levels do not correlate with the changes in DRM in the RNA level. Fluorescence-activated cell sorting (FACS) analysis with parallel cultures, indicated that cells enter the S phase at 1 hour after HU blockade release under these experimental conditions. The experiment was repeated with HEM cells and the results were consistent with previous findings. These data indicate that the level of DRM declines when cells enter the S phase of cell cycle. In order to see the early response of DRM expression just after addition of a mitogen, HEM cells were growth arrested by serum starvation and reintroduced into a synchronous cell division cycle by addition of 10% FCS. By this method, it was shown that biosynthesis of DRM is clearly down-regulated 1.5 hours after serum stimulation.

Several proteins that are involved in the cell cycle regulation are accumulated during starvation such as p27^{Kip1} (76) and cyclin E (86). The pattern of modulation of DRM during the cell cycle was compared with other inhibitors. Whereas p27 tends to accumulate in quiescent cells and declines in response to mitogenic stimulation, p21 levels are generally low in quiescent cells, but rise in response to mitogen treatment.

The pattern of DRM expression during the cell cycle and the first three hours of serum stimulation is very similar to that observed for p27^{Kip1}, but contrasts to p21^{Cip1}. Although the amount of DRM falls significantly during the G₀ to S phase transition, it continues to be synthesized in proliferating cells, leaving the possibility open that its expression might also be regulated periodically.

Previously, it was known that cell cycle regulation of many proteins, such as cyclins, cyclin-dependent kinase inhibitors, p27, occurs via the ubiquitin-proteosome pathway. Also, it has been shown that compared to proliferating cells, quiescent cells contain a far lower amount of p27 ubiquitinating activity (76,77). In order to test a hypothesis that accumulation of DRM in starved cells is also due to increased stability of the protein in quiescent cells, the effect of the proteosome inhibitors, lactocystin (LC) and clasto-lactocystin-β-lactone, and chloroquine, the lysosomal inhibitor was examined.

Degradation of DRM Proteins.

To study the stability and maturation of DRM and monitor the appearance of DRM forms, pulse-chase experiments were performed in primary rat fibroblasts. Cells metabolically labeled with 35S cysteine for 30 min were either lysed immediately (pulse) or incubated in excess of cold cysteine for various periods of time (chase). DRM protein was immunoprecipitated with specific antiserum and immune complexes were separated on SDS-PAGE. Both glycosylated and non-glycosylated forms were detected after a 30 min pulse. The same bands were visible when the pulse period was shortened to 10 min, indicating that glycosylation takes place during or immediately after biosynthesis. Intensity of the labeled bands rapidly decreased over a two-hour chase period, in agreement with an estimated half-life of about 45-60 min. Both glycosylated and non-glycosylated forms were lost at equivalent rates, indicating that glycosylation did not influence protein stability. A mobility shift of all DRM bands was also observed that was visible after a 30 min chase, suggesting that phosphorylation is involved in degradation. To confirm that the shifted bands were indeed phosphorylated, cell extracts were treated after a 30 min pulse and after a 2.5 h chase period with alkaline phosphatase. All DRM bands were sensitive to this treatment, especially after the 2.5 h chase, as shown by their increased electrophoretic mobility.

To determine which of the endosomal/lysosomal or proteasome pathways was involved in DRM protein degradation, pulse chase experiments were performed in the presence of either chloroquine, a lysosomotrophic protein inhibitor or lactacystin, a specific inhibitor of proteasomal degradation. Protein stability was observed to be increased in the presence of both inhibitors, although the observed relative intensity of the upper and lower bands, as well as their mobility, depended on the inhibitor used. Thus, in the presence of chloroquine, the stability of the glycosylated form was apparently increased, compared to that of untreated cells and of the lower non-glycosylated form. In addition, the mobility of the upper stabilized band was increased, suggesting it may have undergone dephosphorylation. These changes are consistent with the hypothesis that phosphatase activity in lysosomes acts to dephosphorylate DRM during treatment. In contrast, in the presence of lactacystin the stability of the lower non-glycosylated form was increased. Moreover, changes in mobility were not observed, suggesting that phosphorylation of all forms was preserved, possibly as a signal for degradation by proteasomes.

### Example III.

Production of EGFP/DRM Fusion Proteins

The EGFP/DRM fusion encoding nucleic acid (SEQ ID NO:1) was constructed as follows: DRM was PCR amplified using: forward primer: CGGGATCCAGAATGAATCGCACGGCATAC (SEQ ID NO:11) and reverse primer: GCGGATCCTTAATCCAAGTCGATGGATATGC (SEQ ID NO:12). The PCR product was digested with BamHI and EcoRI and ligated in frame into the pEGFP-C1 vector digested with BglII and EcoRI. The EGFPC1 coding region is nucleotides 3954-4688 and the DRM coding region is nucleotides 4689-5243. The amino acid sequence of the EGFP/DRM fusion protein is SEQ ID NO:29.

The NUCLEAR LOCALIZATION MUTANT #1(NLS#1), which contains a deletion of the 3′ NLS region of DRM was made by cutting the EGFP/DRM fusion gene (SEQ ID NO:1) with BstXI and ligating in the double stranded synthetic oligonucleotide:

TAAGTCGCTTCGACGTACATTCAGCGA (SEQ ID NO:13) to remove the 3′ portion of the drm gene including the 3′ nuclear localization signal (NLS#1) but leaving the 5′ nuclear localization signal (NLS#2). The EGFP coding region is nucleotides 3954-4688 and the drm N1 mutation coding region is nucleotides 4689-5147. The resulting nucleic acid sequence is SEQ ID NO:5. The amino acid sequence of the NLS#1 mutant is SEQ ID NO:30.

The NUCLEAR LOCALIZATION MUTANT #2 (NLS#2), an EGFP-DRM double mutant, contains a deletion of the 3′ NLS#1 and a point mutation within the upstream NLS#2. The EGFP coding region is nucleotides 613-1338 and the drm 2nls mutant coding region is nucleotides 1339-1815. This mutant was generated by PCR amplification of drm with the 5′ oligonucleotide: AGGAATTCAATGAATCGCACGGCATAC (SEQ ID NO:14) and the 3′ reverse oligonucleotide primer: ACGGGATCCTTACATGGTGGTGAATACTTGGG (SEQ ID NO: 15), which introduces a point mutation in the 5′ NLS#2, rendering it non-functional. The resulting nucleic acid sequence is SEQ ID NO:6 and the amino acid sequence of the NLS#2 mutant is SEQ ID NO:31. This PCR-generated fragment was digested with restriction enzymes BamHI and EcoRI and ligated into a BamHI and EcoRI digested EGFP-C1 vector obtained from Clontech Inc.

Generation of D5del Versions of EGFP-DRM and NLS Mutants:

D5del: The EGFP-DRM nucleotide sequence (SEQ ID NO:1) was digested with BsrGI and Bpu1102I. The double stranded synthetic oligonucleotide:

| | |
|---|---|
| GTACAAGTCCGGACTCAGAATGAGGGCTTCAGGCCT | (SEQ ID NO:16) |
| GAGTCTTACTCCCGAGT | |

was ligated into the digested plasmid producing a EGFP-drm fusion minus the transmembrane domain. The EGFP coding region is nucleotides 3954-4682 and the drm coding region is nucleotides 4683-5129. The resulting nucleic acid is SEQ ID NO:7 and the amino acid sequence of the D5del mutant is SEQ ID NO:32.

NLS#1D5del: The EGFP-NLS#1 mutant nucleotide sequence (SEQ ID NO:5) was digested with BsrGI and Bpu1102I. The double stranded synthetic oligonucleotide:

| | |
|---|---|
| GTACAAGTCCGGACTCAGAATGAGGGCTTCAGGCCT | (SEQ ID NO:17) |
| GAGTCTTACTCCCGAGT | |

was ligated into the digested plasmid producing a EGFP-drm fusion minus the 2nd nuclear localization signal (NLS#2) and the transmembrane domain. The EGFP coding region is nucleotides 3954-4682 and the drm NLS#1D5del mutant coding region is nucleotides 4683-5033. The resulting nucleic acid sequence is SEQ ID NO:8 and the amino acid sequence of the NLS#1D5del mutant is SEQ ID NO:33.

NLS#2D5del: EGFP-NLS#2 mutant nucleotide sequence (SEQ ID NO:6) was digested with BsrGI and Bpu1102I. The double stranded synthetic oligonucleotide:

| | |
|---|---|
| GTACAAGTCCGGACTCAGAATGAGGGCTTCAGGCCT | (SEQ ID NO:18) |
| GAGTCTTACTCCCGAGT | |

was ligated into the digested plasmid producing an EGFP-DRM fusion minus the 1st and 2nd nuclear localization signals and the transmembrane domain. The EGFP coding region is nucleotides 3954-4682 and the DRM nls2\tm mutant coding region is nucleotides 4683-5033. The resulting nucleic acid is SEQ ID NO:9 and the amino acid sequence of the NLS#2D5del mutant is SEQ ID NO:34.

DAvaI: The EGFP-DRM nucleotide sequence (SEQ ID NO:1) was digested with AvaI and the synthetic ds oligonucleotide:

CCGGGGACGAGGACAGCTGTAATTA CCTGCTCCT GTC GACATTAATGGCC (SEQ ID NO:10)

was ligated in, introducing a stop codon at base 4878 in the EGFP/DRM sequence. The resulting nucleic acid sequence is SEQ ID NO:19 and the amino acid sequence of the DAvaI mutant is SEQ ID NO:35.

Although the present process has been described with reference to specific details of certain embodiments thereof, it is not intended that such details should be regarded as limitations upon the scope of the invention except as and to the extent that they are included in the accompanying claims.

Throughout this application, various publications are referenced. The disclosures of these publications in their entireties are hereby incorporated by reference into this application in order to more fully describe the state of the art to which this invention pertains.

### REFERENCES

1. Athanasiou, M., G. Mavrothalassitis, C. C. Yuan, and D. G. Blair. 1996. The gag-myb-ets fusion oncogene alters the apoptotic response and growth factor dependence of interleukin-3 dependent murine cells. Oncogene 12:337-344.

2. Barnes, J. L., and S. Milani. 1995. In situ hybridization in the study of the kidney and renal diseases. Seminars in nephrology, v. 15, No. 1:9-28.

3. Blair, D. G., M. A. Hull, and E. A. Finch. 1979. The isolation and preliminary characterization of temperature sensitive transformation mutants of Moloney Sarcoma Virus. Virology 95:303-316.

4. Boowmeester, T., S. H. Kim, Y. Sasai, B. Lu, and E. M. De Robertis. 1996. Cerberus is a head-inducing secreted factor expressed in the anterior endoderm of spemann's organizer. Nature 382:595-601.

5. Boyd, J. M., S. Malstrom, T. Subramanian, L. K. Venkatesh, U. Schaeper, B. Elangovan, C. D'Sa-Eipper, and G. Chinnadurai. 1994. Adenovirus E1B 19 kDa and Bcl-2 proteins interact with a common set of cellular proteins. Cell 79:341-351.

6. Brody, J. S., and M. C. Williams. 1992. Pulmonary alveolar epithelial cell differentiation. Ann. Rev. Physiol. 54:351-371.

7. Chomczynski, P., and N. Sacchi. 1987. Single-step method of RNA isolation by acid guanidium thiocyanate-phenol-chloroform extraction. Anal. Biochem. 162:156-159.

8. Contente, S., K. Kenyon, D. Rimoldi, and R. M. Friedman. 1990. Expression of gene rrg is associated with reversion of NIH3T3 transformed by LTR-c-H-ras. Science 249:797-798.

9. Denhardt, D. T. 1966. A membrane-filter technique for the detection of complementary DNA. Biochem. Biophys. Res. Commun. 23:641-646.

10. Enomoto, H., T. Ozaki, E. I. Takahashi, N. Nomura, S. Tabata, H. Takahashi, N. Ohnuma, M. Tanabe, J. Iwai, M. Yoshida, T. Matsunaga, and S. Sakiyama. 1994. Identification of human DAN gene, mapping to the putative neuroblastoma tumor suppressor locus. Oncogene 9:2785-2791.

11. Genetic Computer Group. 1994. Program manual for the Wisconsin GCG package. Version 8.0, University of Wisconsin, Madison.

12. Gillet, G., M. Guerin, A. Trembleau, and G. Brun. 1995. A BCL-2 related gene is activated in avian cells transformed by the Rous sarcoma virus. EMBO J. 14:1372-1381.

13. Glück, U., D. J. Kwiatkowski, and A. Ben-Ze'ev. 1993. Suppression of tumorigenicity in simian virus 40-transformed 3T3 cells transfected with α-actinin cDNA. Proc. Natl. Acad. Sci. USA 90:383-387.

14. Gordon, J. I., and M. L. Hermiston. 1994. Differentiation and self-renewal in the mouse gastrointestinal epithelium. Curr. Opin. Cell Biol. 6:795-803.

15. Gross-Bellard, M., P. Oudet, and P. Chambon. 1973. Isolation of high-molecular-weight DNA from mammalian cells. Eur. J. Biochem. 36:32-38.

16. Gum, J. R., J. W. Hicks, N. W. Toribara, E-M. Rothe, R. E. Lagace, and Y. S. Kim. 1992. The human MUC2 intestinal mucin has cysteine-rich subdomains located both upstream and downstream of its central repetitive region. J. Biol. Chem. 267:21375-21383.

17. Hall, P. A., P. J. Coates, B. Ansam, and D. Hopwood. 1994. Regulation of cell number in the mammalian gastrointestinal tract: the importance of apoptosis. J. Cell Sci. 107:3569-3577.

18. Hamelin, R., B. L. Brizzard, M. A. Nash, E. C. Murphy, and R. B. Arlinghaus. 1985. Temperature-sensitive viral RNA expression in ts110 Moloney murine sarcoma virus-infected cells. J. Virol. 50:478-488.

19. Harada, H., M. Kitayawa, N. Tanaka, H. Yamamoto, K. Horada, M. Ishihara, and T. Taniguchi. 1993. Anti-oncogenic and oncogenic potentials of interferon regulation factors-1 and -2. Science 259:971-974.

20. Houle, B., C. Rochette-Egly, and W. E. C. Bradley. 1993. Tumor suppressive effect of the retinoic acid receptor β in human epidermoid lung cancer cells. Proc. Natl. Acad. Sci. USA 90:985-989.

21. Katzov, S., D. Martin-Zanca, and M. Barbacid. 1989. Vav, a novel human oncogene derived from a locus ubiquitously expressed in hematopoietic cells. EMBO J. 8:2283-2290.

22. Kozak, M. 1987. An analysis of 5′-noncoding sequences from 699 vertebrate messenger RNAs. Nucleic Acids Res. 15:8125-8133.

23. Kozak, M. 1992. Regulation of translation in eukaryotic systems. Ann. Rev. Cell Biol. 8:197-225.

24. Levine, A. 1993. The tumor suppressor genes. Ann. Rev. Biochem. 62:623-651.

25. Liang, P., and A. B. Pardee. 1992. Differential display of eukaryotic messenger RNA by means of the polymerase chain reaction. Science 247:967-971.

26. Lin, X., P. J. Nelson, B. Frankfort, E. Tombler, R. Johnson, and J. H. Gelman. 1995. Isolation and characterization of a novel mitogenic regulatory gene, 322, which is transcriptionally suppressed in cells transformed by src and ras. Mol. Cell. Biol. 15:2754-2762.

27. Nuygen, M., P. E. Branton, P. A. Walton, Z. N. Oltvai, S. J. Korsmeyer, and G. C. Shore. 1994. Role of membrane anchor domain of Bcl-2 in suppression of apoptosis caused by E1B-defective adenovirus. J. Biol. Chem. 269:16521-16524.

28. Ozaki, T., and S. Sakiyama. 1993. Molecular cloning and characterization of a cDNA showing negative regulation in v-src-transformed 3Y1 rat fibroblasts. Proc. Natl. Acad. Sci. USA 90:2593-2597.

29. Ozaki, T., and S. Sakiyama. 1994. Tumor-suppressive activity of NO3 gene product in v-src-transformed Rat 3Y1 fibroblasts. Cancer Res. 54:646-648.

30. Ozaki, T., Y. Nakamura, H. Enomoto, M. Hirose, and S. Sakiyama. 1995. Overexpression of DAN gene product in normal rat fibroblasts causes a retardation of the entry into the S phase. Cancer Res. 55:895-900.

31. Prasad, G. L., R. A. Fuldner, and H. L. Cooper. 1993. Expression of transduced tropomyosin 1 cDNA suppresses neoplastic growth of cells transformed by the ras oncogene. Proc. Natl. Acad. Sci. USA 90:7039-7043.

32. Preisig, P. A., and H. A. Franch. 1995. Renal epithelial cell hyperplasia and hypertrophy. Seminars in nephrology 15(4):327-340.

33. Rao, L., M. Debbas, P. Sabbatini, D. Hockenbery, S. Korsmeyer, and E. White. 1992. The adenovirus E1A proteins induce apoptosis, which is inhibited by the E1B 19 kDa and Bcl-2 proteins. Proc. Natl. Acad. Sci. USA 89:7742-7746.

34. Sager, R. 1989. Tumor suppressor genes: the puzzle and the promise. Science 246:1406-1412.

35. Sambrook, J., E. Fritsch, and T. Maniatis. 1989. Molecular cloning: A laboratory manual, 2nd Ed., Cold Spring Harbor Laboratory, Cold Spring Harbor, N.Y.

36. Sanger, F. 1981. Determination of nucleotide sequences in DNA. Science 214:1205-1210.

37. Sassoon, D., and N. Rosenthal. 1993. Methods Enzymol. 225:389-403.

38. Shih, C., and R. A. Weinberg. 1982. Isolation of a transforming sequence from a human bladder carcinoma cell line. Cell 29:161-169.

39. Sprague, J., J. H. Condra, H. Arnheiter, and R. A. Lazzarini. 1983. Expression of a recombinant DNA gene coding for the resiculor stomatitis virus nucleocapsid protein. J. Virol. 45: 773-781.

40. Topol, L. Z., A. G. Tatosyan, D. Blair, and F. L. Kisselov. 1991. A new recipient line for the transfection of biologically active oncogenes. Mol. Biol. (Translated) 25(2):541-551.

41. Topol, L. Z., M. Marx, G. Calothy, and D. G. Blair. 1995. Transformation-resistant mos revertant is unable to activate MAP kinase in response to v-mos or v-raf. Cell Growth Differ. 6:27-38.

42. Topol, L. Z., and D. G. Blair. 1995. Activation of the mitogen-activated protein kinase cascade in response to the temperature inducible expression of v-mos kinase. Cell Growth Differ. 6:1119-1127.

43. White, E., P. Sabbatini, M. Debbas, W. S. M. Wold, D. I. Kusher, and L. Gooding. 1992. The 19-kilodalton adenovirus E1B transforming protein inhibits programmed cell death and prevents cytolysis by tumor necrosis factor α. Mol. Cell. Biol. 12:2570-2580.

44. Zou, Z., A. Anisowicz, M. J. C. Hendrix, A. Thor, M. Neveu, S. Sheng, K. Rafidi, E. Seftor, and R. Sager. 1994. Maspin, a serpin with tumor-suppressing activity in human mammary epithelial cells. Science 263:526-529.

45. Lesser M L. Design and implementation of clinical trials. In: Statistics in Medical Research—Methods and Issues with Applications in Cancer Research. Ed: Mike V and Stanley K F, New York, Wiley. 1982.

46. Gehan E A, Schneiderman M A: Experimental Design of Clinical Trials, in Holland J F and Frei E, Ill, eds. Cancer Medicine (2nd ed.). Lea and Febinger, Philadelphia, 531-553,1982.

47. Gail M, Gart J J: The Determination of Sample Sizes for Use with the Exact Conditional Test in 2×2 Comparative Trials. Biometrics, 29, 441-448, 1973.

48. Lee E T: Statistical Methods for Survival Data Analysis, Wiley, New York, 1992.

49. Kalbfleisch J D, Prentice R L: The Statistical Analysis of Failure Time Data, New York, Wiley, 1980.

50. Pastan et al. “A retrovirus carrying an MDR1 cDNA confers multidrug resistance and polarized expression of P-glycoprotein in MDCK cells.” Proc. Nat. Acad. Sci. 85:4486 (1988)

51. Miller et al. “Redesign of retrovirus packaging cell lines to avoid recombination leading to helper virus production.” Mol. Cell Biol. 6:2895 (1986).

52. Mitani et al. “Transduction of human bone marrow by adenoviral vector.”Human Gene Therapy 5:941-948 (1994).

53. Goodman et al. “Recombinant adeno-associated virus-mediated gene transfer into hematopoietic progenitor cells.” Blood 84:1492-1500 (1994)

54. Naidini et al. “In vivo gene delivery and stable transduction of nondividing cells by a lentiviral vector.” Science 272:263-267 (1996))

55. Agrawal et al. “Cell-cycle kinetics and VSV-G pseudotyped retrovirus mediated gene transfer in blood-derived CD34⁺ cells.” Exp. Hematol. 24:738-747 (1996).

56. Schwarzenberger et al. “Targeted gene transfer to human hematopoietic progenitor cell lines through the c-kit receptor.” Blood 87:472-478 (1996).

57. Fields, et al. (1990) Virology, Raven Press, New York.

58. Michieli, P., Li, W., Lorenzi, M. V., Miki, T., Zakut, R., Givol, D., and Pierce, J. H. (1996) Oncogene 12, 775-784.

59. Crystal, R. G. 1997. Phase I study of direct administration of a replication deficient adenovirus vector containing E. coli cytosine deaminase gene to metastatic colon carcinoma of the liver in association with the oral administration of the pro-drug 5-fluorocytosine. Human Gene Therapy 8:985-1001.

60. Alvarez, R. D. and D. T. Curiel. 1997. A phase I study of recombinant adenovirus vector-mediated delivery of an anti-erbB-2 single chain (sFv) antibody gene from previously treated ovarian and extraovarian cancer patients. Hum. Gene Ther. 8:229-242.

61. Lewin, “Genes V” Oxford University Press Chapter 7, pp. 171-174 (1994).

62. Sambrook et al., Molecular Cloning. A Laboratory Manual. 2nd Ed., Cold Spring Harbor Laboratory, Cold Spring Harbor, N.Y. (1989).

63. Lewin, “Genes V” Oxford University Press Chapter 1, pp. 9-13 (1994).

64. Kunkel et al., Methods Enzymol. 154:367 (1987).

65. Topol, L Z, Marx, M, Laugier, D, Bogdanova, N N, Boubnov, N V, Clausen, P A, Calothy, G and Blair, D G. 1997. Identification of drm, a novel gene whose expression is suppressed in transformed cells and which can inhibit growth of normal but not transformed cells in culture. Mol. Cell Biol. 17:4801-4810.

66. Yonish-Rouach E, Resvitzky D, Lotem J, Sachs L, Kimchi A and Oren M. 1991. Wild-type p53 induces apoptosis of myeloid leukemia cells that is inhibited by interleukin-6. Nature 352:345-347.

67. Goldstein S. 1990. Replicative senescence: the human fibroblast comes of age. Science 249:1129-1133.

68. Schneider C, King R M and Philipson L. 1988. Genes specifically expressed at growth arrest of mammalian cells. Cell 54:787-793.

69. Del Sal G, Ruaro M E, Philipson L and Schneider C. 1992. The growth arrest-specific gene gas1 is involved in growth suppression. Cell 70:595-607.

70. Brancolini C, Bottega S and Schneider C. 1992. Gas 2, a growth arrest-specific protein, is a component of the microfilament network system. Journal of Cell Biology 117:1251-1261.

71. Sagesaka T, Boubnov N, Okuyama T, Paulus H and Sarkar N. 1994. Deoxyribonucleic acid replication in fetal cells. American Journal of Obstetrics and Gynecology 170:468-473.

72. Topol L Z, Marx M, Laugier D, Bogdanova N N, Boubnov N V, Clausen P A, Calothy G and Blair D G. 1997. Identification of drm, a novel gene whose expression is suppressed in transformed cells and which can inhibit growth of normal but not transformed cells in culture. Molecular and Cellular Biology 17:4801-4810.

73. Gaypay G, Schmitt K, Fizames C, Jones M, Vega-Ozarny N, Spillet D, Muselet D, Prud'Homme J-F, Dib C, Auffray C, Morisette J, Weissenbach J and Goodfellow P N. 1996. A radiation hybrid map of the human genome. Human Molecular Genetics 5:339-346.

74. Kozak M. 1991. Structure features in eukaryotic mRNAs that modulate the initiation of translation. Journal of Biological Chemistry 266:19867-19870.

75. Dib C, Fauré S, Fizames C, Samson D, Drouot N, Vignal A, Millasseau P, Marc S, Hazan J, Seboun E, Lathrop M, Gyapay G, Morisette J and Weissenbach J. 1996. A comprehensive genetic maps of the human genome based on 5,264 microsatellites. Nature 380:152-154.

76. Pagano M, Tam S W, Theodoras A M, Beer-Romero P, Del Sal G, Chan V, Yew P R, Draetta G F and Rolfe M. 1995. Role of the ubiquitin-proteosome pathway in regulating abundance of the cyclin-dependent kinase inhibitor p27. Science 269:682-685.

77. Alessandrini A, Chiaur D S and Pagano M. 1992. Regulation of the cyclin-dependent kinase inhibitor p27 by degradation and phosphorylation. Leukemia 11:342-345.

78. Koff A, Cross F, Fisher A, Schumacher J, Leguellee K, Philippe M and Roberts J M. 1991. Human cyclin E, a new cyclin that interacts with two members of the CDC2 gene family. Cell 66:1217-1228.

79. Forss-Petter S, Danielson P and Sutcliffe J G. 1986. Neuron-specific Enolase: Complete structure of rat mRNA, multiple transcriptional start sites and evidence suggesting post-transcriptional control. Journal of Neuroscience Research 16:141-156

80. Spector D L, Fu X-D and Maniatis T. 1991. Associations between distinct pre-mRNA splicing components and the cell nucleus. EMBO J. 10:3467-3481.

81. Huang S, Deerinch J, Ellisman M and Spector D L. 1994. In vivo analysis of the stability and transport of nuclear Poly(A)⁺ RNA. Journal of Cell Biology 126: 878-899.

82. Stauber R H, Horie K, Carney P, Hudson E A, Tarasova N I, Gaitanaris G A and Pavlakis G N. 1998. Development and applications of enhanced green fluorescent protein mutants. BioTechniques 24:462-471.

83. Forss-Petter S, Danielson P and Sutcliffe J G. 1986. Neuron-specific Enolase: Complete structure of rat mRNA, multiple transcriptional start sites and evidence suggesting post-transcriptional control. Journal of Neuroscience Research 16:141-156.

84. Tohyama T, Lee V M-Y and Trojanovski J. 1993. Co-expression of low molecular weight neurofilament protein and glial fibrillary acidic protein in established human glioma cell lines. American Journal of Pathology 142:883-892.

85. Pagano M, Tam S W, Theodoras A M, Beer-Romero P, Del Sal G, Chau V, Yew P R, Draetta G F and Rolfe M. 1995. Role of the ubiquitin-proteosome pathway in regulating abundance of the cyclin-dependent kinase inhibitor p27. Science 269:682-685

86. Rolfe M, Chin M I and Pagano M. 1997. The ubiquitin-mediated proteolytic pathway as a therapeutic area. Journal of Molecular medicine 75:8-17.

87. Lee M, Larner J M and Hamlin J L. 1997. Cloning and characterization of Chinese hamster p53 cDNA. Gene 184:177-183.

88. Brake A J, Merryweather J P, Coit D G, Heberlein U A, Masiarz F R, Mullenbach G T, Urdea M S, Valenzuela P, and Barr P J 1984. Alpha-factor-directed synthesis and secretion of mature foreign proteins in Saccharomyces cerevisiae, PNAS 82:4642-4646.

89. Sternsdorf, T., Jensen, K., Zuchner, D. and Will, H. 1997. Cellular localization, expression, and structure of the nuclear dot protein 52. J. Cell Biol. 138: 435-448.

90. Harlow and Lane. Antibodies, A Laboratory Manual. Cold Spring Harbor Laboratory, Cold Spring Harbor, N.Y., 1988.

91. Martin E W: Remington's Pharmaceutical Sciences, latest ed., Mack Publishing Co., Easton, Pa.

TABLE 1
Drm IS PREFERENTIALLY EXPRESSED IN TERMINALLY-
DIFFERENTIATED CELLS

| Tissue | Cell type | Proliferation/Differentiation |
|---|---|---|
| Brain | Neuron | non/terminally |
| | Glial | low/diff. |
| Kidney | Tubular | low/diff. |
| | epithelial | |
| Lung | Type 1 | none/terminally |
| | epithelial | |
| Intestine | Goblet | low/diff. |
| Spleen | Megakaryocyte | diff. |

TABLE 2
DRM Expression in Normal and Malignant Cell Lines

| | Screened Amount | Amount With |
|---|---|---|
| | of Cell Lines | Positive Expression |
| Normal Cell Lines | | |
| Diploid fibroblasts | 10 | 10 |
| Normal astrocytes | 1 | 1 |
| Normal melanocytes | 1 | 0 |
| Normal mammary gland | 1 | 0 |
| HUVEC | 1 | 0 |
| Malignant Cell Lines | | |
| Adenocarcinoma | 21 | 0 |
| Fibrosarcoma | 3 | 0 |
| Sarcoma | 5 | 0 |
| Melanoma | 5 | 0 |
| Carcinoma | 10 | 0 |
| Astrocytoma | 1 | 0 |
| Rhabdomyosarcoma | 1 | 0 |

What is claimed is:

1. An isolated nucleic acid having the nucleotide sequence of SEQ ID NO:2.

2. An isolated polypeptide having the amino acid sequence of SEQ ID NO:36.

3. An isolated nucleic acid encoding the polypeptide of claim 2.

4. An isolated nucleic acid having the nucleotide sequence of SEQ ID NO:3.

5. An isolated nucleic acid having the nucleotide sequence of SEQ ID NO:4.

6. A fragment of DRM protein comprising the amino acid sequence encoded by nucleotides 4689 through 5243 of SEQ ID NO: 1.

7. An isolated nucleic acid encoding the amino acid sequence of claim 6.

8. A fragment of DRM protein comprising the amino acid sequence encoded by nucleotides 4683 through 5147 of SEQ ID NO: 5.

9. An isolated nucleic acid encoding the amino acid sequence of claim 8.

10. A fragment of DRM protein comprising the amino acid sequence encoded by nucleotides 1339 through 1815 of SEQ ID NO: 6.

11. An isolated nucleic acid encoding the amino acid sequence of claim 10.

12. A fragment of DRM protein comprising the amino acid sequence encoded by nucleotides 4683 through 5129 of SEQ ID NO: 7.

13. An isolated nucleic acid encoding the amino acid sequence of claim 12.

14. A fragment of DRM protein comprising the amino acid sequence encoded by nucleotides 4683 through 5033 of SEQ ID NO: 8.

15. An isolated nucleic acid encoding the amino acid sequence of claim 14.

16. A fragment of DRM protein comprising the amino acid sequence encoded by nucleotides 4689 through 5243 of SEQ ID NO: 19, wherein a stop codon is introduced at nucleotide 4878 of SEQ ID NO: 19.

17. An isolated nucleic acid encoding the amino acid sequence of claim 16.
